SEQUENCE LISTING (28 sequences)

SEQ ID NO: 1 - protein, 119 aa, Rattus norvegicus
  MISC_FEATURE (26)..(35): CDRH1
EVKLVESGGGLEQPKGSLKLSCTVSGFDFNSYGMSWVRQAPGKGLDLVADINSKSYNYATYYADSVKDRFTISRDDSQSMVYLEMDNLKTEDTALYYCTVHHSDYFEYWGQGVMVTVSS

SEQ ID NO: 2 - protein, 109 aa, Rattus norvegicus
  MISC_FEATURE (24)..(34): CDRL1
DIQMTQSPASLSASLGETVSIECLASEDIYNNLAWYQQKPGKSPQLLIYHASSLQDGVPSRFSGSGSGTQYSLKINSLESEDAATYFCLQDSEYPPTFGGGTKLELKRT

SEQ ID NO: 3 - protein, 10 aa, Rattus norvegicus
GFDFNSYGMS

SEQ ID NO: 4 - protein, 19 aa, Rattus norvegicus
DINSKSYNYATYYADSVKD

SEQ ID NO: 5 - protein, 8 aa, Rattus norvegicus
HHSDYFEY

SEQ ID NO: 6 - protein, 11 aa, Rattus norvegicus
LASEDIYNNLA

SEQ ID NO: 7 - protein, 7 aa, Rattus norvegicus
HASSLQD

SEQ ID NO: 8 - protein, 9 aa, Rattus norvegicus
LQDSEYPPT

SEQ ID NO: 9 - protein, 629 aa, Homo sapiens [sequence omitted]

SEQ ID NO: 10 - protein, 662 aa, Homo sapiens
  SIGNAL (1)..(24)
MEPLVTWVVPLLFLFLLSRQGAACRTSECCFQDPPYPDADSGSASGPRDLRCYRISSDRYECSWQYEGPTAGVSHFLRCCLSSGRCCYFAAGSATRLQFSDQAGVSVLYTVTLWVESWARNQTEKSPEVTLQLYNSVKYEPPLGDIKVSKLAGQLRMEWETPDNQVGAEVQFRHRTPSSPWKLGDCGPQDDDTESCLCPLEMNVAQEFQLRRRQLGSQGSSWSKWSSPVCVPPENPPQPQVRFSVEQLGQDGRRRLTLKEQPTQLELPEGCQGLAPGTEVTYRLQLHMLSCPCKAKATRTLHLGKMPYLSGAAYNVAVISSNQFGPGLNQTWHIPADTHTEPVALNISVGTNGTTMYWPARAQSMTYCIEWQPVGQDGGLATCSLTAPQDPDPAGMATYSWSRESGAMGQEKCYYITIFASAHPEKLTLWSTVLSTYHFGGNASAAGTPHHVSVKNHSLDSVSVDWAPSLLSTCPGVLKEYVVRCRDEDSKQVSEHPVQPTETQVTLSGLRAGVAYTVQVRADTAWLRGVWSQPQRFSIEVQVSDWLIFFASLGSFLSILLVGVLGYLGLNRAARHLCPPLPTPCASSAIEFPGGKETWQWINPVDFQEEASLQEALVVEMSWDKGERTEPLEKTELPEGAPELALDTELSLEDGDRCKAKM

SEQ ID NO: 11 - protein, 119 aa, Artificial Sequence
  Rat CDRs and human frameworks
QVQLVESGGGVVQPGRSLRLSCAASGFDFNSYGMSWVRQAPGKGLEWVADINSKSYNYATYYADSVKDRFTISRDNSKNTLYLQMNSLRAEDTAVYYCARHHSDYFEYWGQGTLVTVSS

SEQ ID NO: 12 - protein, 109 aa, Artificial Sequence
  Rat CDRs and human frameworks
DIQMTQSPSSLSASVGDRVTITCLASEDIYNNLAWYQQKPGKAPKLLIYHASSLQDGVPSRFSGSGSGTDFTLTISSLQPEDFATYYCLQDSEYPPTFGQGTKVEIKRT

SEQ ID NO: 13 - protein, 119 aa, Artificial Sequence
  Rat CDRs and modified human frameworks
QVQLVESGGGVVQPGRSLRLSCAASGFDFNSYGMSWVRQAPGKGLDLVADINSKSYNYATYYADSVKDRFTISRDNSKNTLYLQMNSLRAEDTAVYYCAVHHSDYFEYWGQGTLVTVSS

SEQ ID NO: 14 - protein, 109 aa, Artificial Sequence
  Modified rat CDRs and modified human frameworks
DIQMTQSPSSLSASVGDRVTITCRASQDIYTNLAWYQQKPGKAPKLLIYHASSLQDGVPSRFSGSGSGTDYTLTISSLQPEDFATYYCLQDSEYPPTFGQGTKVEIKRT

SEQ ID NO: 15 - protein, 11 aa, Artificial Sequence
  Modified rat sequence
RASQDIYTNLA

SEQ ID NO: 16 - protein, 449 aa, Artificial Sequence
  Rat CDRs, modified human frameworks, and human IgG1 constant domain
QVQLVESGGGVVQPGRSLRLSCAASGFDFNSYGMSWVRQAPGKGLDLVADINSKSYNYATYYADSVKDRFTISRDNSKNTLYLQMNSLRAEDTAVYYCAVHHSDYFEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 214 aa, Artificial Sequence
  Modified rat CDRs, modified human frameworks, and human kappa constant domain
DIQMTQSPSSLSASVGDRVTITCRASQDIYTNLAWYQQKPGKAPKLLIYHASSLQDGVPSRFSGSGSGTDYTLTISSLQPEDFATYYCLQDSEYPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 19 aa, Mus musculus
MAVLGLLFCLVTFPSCVLS

SEQ ID NO: 19 - protein, 19 aa, Rattus norvegicus
MAPVQLLGLLVLFLPAMRC

SEQ ID NO: 20 - protein, 10 aa, Artificial Sequence
  Modified rat sequence
GFDFSSYGMS

SEQ ID NO: 21 - protein, 7 aa, Artificial Sequence
  Modified rat sequence
HASSLQS

SEQ ID NO: 22 - protein, 119 aa, Artificial Sequence
  Modified rat CDRs and modified human frameworks
QVQLVESGGGVVQPGRSLRLSCAASGFDFSSYGMSWVRQAPGKGLDLVADINSKSYNYATYYADSVKDRFTISRDNSKNTLYLQMNSLRAEDTAVYYCAVHHSDYFEYWGQGTLVTVSS

SEQ ID NO: 23 - protein, 109 aa, Artificial Sequence
  Modified rat CDRs and modified human frameworks
DIQMTQSPSSLSASVGDRVTITCRASQDIYTNLAWYQQKPGKAPKLLIYHASSLQSGVPSRFSGSGSGTDYTLTISSLQPEDFATYYCLQDSEYPPTFGQGTKVEIKRT

SEQ ID NO: 24 - protein, 449 aa, Artificial Sequence
  Modified rat CDRs, modified human frameworks, and human IgG1 constant domain
QVQLVESGGGVVQPGRSLRLSCAASGFDFSSYGMSWVRQAPGKGLDLVADINSKSYNYATYYADSVKDRFTISRDNSKNTLYLQMNSLRAEDTAVYYCAVHHSDYFEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 214 aa, Artificial Sequence
  Modified rat CDRs, modified human frameworks, and human kappa constant domain
DIQMTQSPSSLSASVGDRVTITCRASQDIYTNLAWYQQKPGKAPKLLIYHASSLQSGVPSRFSGSGSGTDYTLTISSLQPEDFATYYCLQDSEYPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 446 aa, Artificial Sequence
  Modified rat CDRs, modified human frameworks, and modified human IgG4 constant domain
QVQLVESGGGVVQPGRSLRLSCAASGFDFSSYGMSWVRQAPGKGLDLVADINSKSYNYATYYADSVKDRFTISRDNSKNTLYLQMNSLRAEDTAVYYCAVHHSDYFEYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 27 - DNA, 1395 bp, Artificial Sequence
  Mouse signal sequence, modified rat CDRs, modified human frameworks, modified human IgG4 constant domain
atggccgtgctgggcctgctgttctgcctggtgaccttcccttcctgcgtgctgtcccaggtgcagctggtcgagtctggcggcggtgtggtgcagcctggccggtccctgagactgtcctgcgccgcctccggcttcgacttctcctcctacggcatgtcctgggtgcgccaggctcctggcaagggcctcgacctggtggccgacatcaactccaagtcctacaactacgccacctactacgccgactccgtgaaggaccggttcaccatctcccgggacaactccaagaacaccctgtacctgcagatgaactccctgcgggccgaggacaccgccgtgtactactgcgccgtgcaccactccgactacttcgagtactggggccagggcaccctggtgaccgtgtcctccgctagcaccaagggcccttccgtgttccctctggccccttgctcccggtccacctccgagtccaccgccgctctgggctgtctggtgaaggactacttccctgagcctgtgacagtgtcctggaactctggcgccctgacctccggcgtgcacaccttccctgccgtgctgcagtcctccggcctgtactccctgtcctccgtggtgacagtgccttcctcctccctgggcaccaagacctacacctgcaacgtggaccacaagccttccaacaccaaggtggacaagcgggtggagtccaagtacggccctccttgccctccctgccctgcccctgagttcctgggcggaccctccgtgttcctgttccctcctaagcctaaggacaccctgatgatctcccggacccctgaagtgacctgcgtggtggtggatgtgtcccaggaagatcctgaggtccagttcaattggtacgtggacggcgtggaggtgcacaacgccaagaccaagcctcgggaggaacagttcaactccacctaccgggtggtgtccgtgctgaccgtgctgcaccaggactggctgaacggcaaagaatacaagtgcaaggtgtccaacaagggcctgccctcctccatcgaaaagaccatctccaaggccaagggccagcctcgcgagcctcaggtgtacaccctgcctccctctcaagaagagatgaccaagaaccaggtgtccctgacatgcctggtgaagggcttctacccttccgatatcgccgtggagtgggagtccaacggccagcctgagaacaactacaagaccacccctcctgtgctggactccgacggctccttcttcctgtactccaggctgaccgtggacaagtcccggtggcaggaaggcaacgtcttttcctgctccgtgatgcacgaggccctgcacaaccactacacccagaagtccctgtccctgtctctgggcaag

SEQ ID NO: 28 - DNA, 699 bp, Artificial Sequence
  Rat signal sequence, modified rat CDRs, modified human frameworks, human kappa constant domain
atggcccctgtgcagctgctgggcctgctggtgctgttcctgcctgccatgagatgcgacatccagatgacccagtccccctcctctctgtccgcctccgtgggcgacagagtgaccatcacctgccgggcctcccaggacatctacaccaacctggcctggtatcagcagaagcctggcaaggcccctaagctgctgatctaccacgcctccagcctgcagtccggcgtgccttcccggttctccggctccggctctggcaccgactacaccctgaccatctcttccctgcagcctgaggacttcgccacctactactgcctgcaggactccgagtaccctcctaccttcggccagggcaccaaggtggagatcaagcgtacggtggccgctccttccgtgttcatcttccctccctccgacgagcagctgaagtccggcaccgcctctgtggtgtgcctgctgaacaacttctaccctcgggaggccaaggtgcagtggaaggtggacaacgccctgcagtctggcaactcccaggaatccgtcaccgagcaggactccaaggactctacctactccctgtcctccaccctgaccctgtccaaggccgactacgagaagcacaaggtgtacgcctgcgaagtgacccaccagggcctgtcctctcccgtgaccaagtccttcaaccggggcgagtgc

(19) United States
(12) Patent Application Publication
(10) Pub. No.: US 2011/0158992 A1
(43) Pub. Date: Jun. 30, 2011

(54) Engineered Anti-IL-23R Antibodies

(75) Inventors: Leonard G. Presta, Palo Alto, CA (US); Grigori P. Ermakov, Palo Alto, CA (US)

(73) Assignee: SCHERING CORPORATION, Kenilworth, NJ (US)

(21) Appl. No.: 13/060,665

(22) PCT Filed: Aug. 25, 2009
(86) PCT No.: PCT/US2009/054844

§ 371 Date: Feb. 24, 2011

Related U.S. Application Data
(60) Provisional application No. 61/092,312, filed on Aug. 27, 2008.
(60) Provisional application No. 61/223,971, filed on Jul. 8, 2009.

(51) Int. Cl. A61K 39/395; C07K 16/28; C07H 21/00; C12N 15/63; C12P 21/02; C12N 5/10; A61P 37/06; A61P 29/00; A61P 19/02; A61P 17/06; A61P 1/00; A61P 25/28; A61P 3/10; A61P 35/00

(52) U.S. Cl. 424/133.1; 530/389.6; 530/387.3; 536/23.53; 435/320.1; 435/69.6; 435/328

(57) ABSTRACT

Antibodies to human IL-23R are provided, as well as uses thereof, e.g. in treatment of inflammatory, autoimmune, and proliferative disorders.

### FIELD OF THE INVENTION

The present invention relates generally to antibodies specific for human interleukin-23R (IL-23R) and their use in treating inflammatory, autoimmune and proliferative disorders.

The Sequence Listing filed electronically herewith is also hereby incorporated by reference in its entirety (File Name: BP06866L02US_SeqListing.txt; Date Created: Jul. 8, 2009; File Size: 44.0 KB.)

### BACKGROUND OF THE INVENTION

The immune system functions to protect individuals from infective agents, e.g., bacteria, multi-cellular organisms, and viruses, as well as from cancers. This system includes several types of lymphoid and myeloid cells such as monocytes, macrophages, dendritic cells (DCs), eosinophils, T cells, B cells, and neutrophils. These lymphoid and myeloid cells often produce signaling proteins known as cytokines. The immune response includes inflammation, i.e., the accumulation of immune cells systemically or in a particular location of the body. In response to an infective agent or foreign substance, immune cells secrete cytokines which, in turn, modulate immune cell proliferation, development, differentiation, or migration. Immune response can produce pathological consequences, e.g., when it involves excessive inflammation, as in the autoimmune disorders. See, e.g., Abbas et al. (eds.) (2000) Cellular and Molecular Immunology, W.B. Saunders Co., Philadelphia, Pa.; Oppenheim and Feldmann (eds.) (2001) Cytokine Reference, Academic Press, San Diego, Calif.; von Andrian and Mackay (2000) New Engl. J. Med. 343:1020-1034; Davidson and Diamond (2001) New Engl. J. Med. 345:340-350.

Interleukin-12 (IL-12) is a heterodimeric molecule composed of p35 and p40 subunits. Studies have indicated that IL-12 plays a critical role in the differentiation of naïve T cells into T-helper type 1 CD4′ lymphocytes that secrete IFNγ. It has also been shown that IL-12 is essential for T cell dependent immune and inflammatory responses in vivo. See. e.g., Cua et al. (2003) Nature 421:744-748. The IL-12 receptor is composed of IL-12Rβ1 and IL-12Rβ2 subunits. See Presky et al. (1996) Proc. Nat'l Acad. Sci. USA 93:14002.

Interleukin-23 (IL-23) is a heterodimeric cytokine comprised of two subunits, p19 which is unique to IL-23, and p40, which is shared with IL-12. The p19 subunit is structurally related to IL-6, granulocyte-colony stimulating factor (G-CSF), and the p35 subunit of IL-12. IL-23 mediates signaling by binding to a heterodimeric receptor, comprised of IL-23R and IL-12Rβ1, which is shared by the IL-12 receptor. See Parham et at (2000) J. Immunol. 168:5699.

A number of early studies demonstrated that the consequences of a genetic deficiency in p40 knockout (“KO”) mice were more severe than those found in a p35KO mouse. Some of these. results were eventually explained by the discovery of IL-23, and the finding that the p40KO prevents expression of not only IL-12, but also of IL-23 (see, e.g., Oppmann et al. (2000) Immunity 13:715-725; Wiekowski et at (2001) J. Immunol. 166:7563-7570; Parham et al, (2002) J. Immunol. 168:5699-708; Frucht (2002) Sci STKE 2002, E1-E3; Elkins et al. (2002) Infection Immunity 70:1936-1948).

Recent studies, through the use of p40 KO mice, have shown that blockade of both IL-23 and IL-12 is an effective treatment for various inflammatory and autoimmune disorders. However, the blockade of IL-12 through p40 appears to have various systemic consequences such as increased susceptibility to opportunistic microbial infections. Bowman et al. (2006) Curr. Opin. Infect. Dis. 19:245.

IL-23R has been implicated as a critical genetic factor in the inflammatory bowel disorders Crohn's disease and ulcerative colitis. Duerr et al. (2006) Science 314:1461. A genome-wide association study found that the gene for IL-23R was highly associated with Crohn's disease, with an uncommon coding variant (Arg381Gln) conferring strong protection against the disease. This genetic association confirms prior biological findings (Yen et al. (2006) J. Clin. Investigation 116:1218) suggesting that IL-23 and its receptor are promising targets for new therapeutic approaches to treating IBD.

Therapeutic antibodies may be used to block cytokine activity. The most significant limitation in using antibodies as a therapeutic agent in vivo is the immunogenicity of the antibodies. As most monoclonal antibodies are derived from rodents, repeated use in humans results in the generation of an immune response against the therapeutic antibody. Such an immune response esuits in a loss of therapeutic efficacy at a minimum and a potential for fatal anaphylactic response. Initial efforts to reduce the immunogenicity of rodent antibodies involved the production of chimeric antibodies, in which mouse variable regions were fused with human constant regions. Liu et al. (1987) Proc. Natl. Acad. Sci. USA 84:3439-43. However, mice injected with hybrids of human variable regions and mouse constant regions develop a strong anti-antibody response directed against the human variable region, suggesting that the retention of the entire rodent Fv region in such chimeric antibodies may still result in unwanted immunogenicity in patients.

It is generally believed that complementarity determining region (CDR) loops of variable domains comprise the binding site of antibody molecules. Therefore, the grafting of rodent CDR loops onto human frameworks (i.e., humanization) was attempted to further minimize rodent sequences. Jones et al. (1986) Nature 321:522; Verhoeyen et al. (1988) Science 239:1534. However, CDR loop exchanges still do not uniformly result in an antibody with the same binding properties as the antibody of origin. Changes in framework residues (FR), residues involved in CDR loop support, in humanized antibodies also are required to preserve antigen binding affinity. Kabat et al. (1991) J. Immunol. 147:1709. While the use of CDR grafting and framework residue preservation in a number of humanized antibody constructs has been reported, it is difficult to predict if a particular sequence will result in the antibody with the desired binding, and sometimes biological, properties. See, e.g., Queen et al. (1989) Proc. Natl. Acad. Sci. USA 86:10029, Gorman et al. (1991) Proc. Natl. Acad. Sci. USA 88:4181, and Hodgson (1991) Biotechnology(NY) 9:421-5.

The need exists for improved methods and compositions for the treatment of inflammatory, autoimmune, and proliferative disorders, e.g. by use of agents that prevent IL-23 signaling through its receptor, such as antagonists of the interaction of IL-23 and the IL-23 receptor. Alternatively such agents could be used to target cells expressing IL-23R for specific ablation. Preferably, such antagonists would have a high affinity for the target molecule, and would be able to block the IL-23 interaction with its receptor at relatively low doses. Preferably, such methods and compositions would be highly specific for IL-23, and not interfere with the activity of other cytokines, such as IL-12. Preferably, such methods and compositions would employ antagonists suitable for modification for the delivery of cytotoxic payloads to target cells, but also suitable for non-cytotoxic uses. Preferably, such methods and compositions would employ antibodies modified to it theirimmunogenicity when administered to a subject in need thereof.

### SUMMARY OF THE INVENTION

The present invention meets these needs in the art and more by providing antagonists of human IL-23R, e.g. humanized anti-human IL-23R antibodies. In particular, the present invention relates to improved humanized anti-human IL-23R antibodies, such as antibody hu8B10-b and antibody hu8B10-c, and variants thereof.

In one aspect the invention provides binding compounds, such as antibodies or fragments thereof, including humanized or chimeric recombinant antibodies, that bind to human IL-23R, comprising an antibody light chain variable domain, or antigen binding fragment thereof, having at least one, two or three CDRs selected from the group consisting of SEQ ID NOs: 6-8, 15 and 21. In one embodiment, the binding compound of the present invention comprises a light chain variable domain comprising a CDRL1 selected from the group consisting of SEQ ID NOs: 6 and 15; a CDRL2 selected from the group consisting of SEQ ID NOs: 7 and 21; and a CDRL3 consisting of SEQ ID NO: 8.

In one embodiment, the binding compound comprises an antibody heavy chain variable domain, or antigen binding fragment thereof, having at least one, two or three CDRs selected from the group consisting of SEQ ID NOs: 3-5 and 20. In one embodiment, the binding compound of the present invention comprises a heavy chain variable domain comprising a CDRH1 selected from the group consisting of SEQ ID NOs: 3 and 20; a CDRH2 consisting of SEQ ID NO: 4; and a CDRH3 consisting of SEQ ID NO: 5.

In other embodiments the binding compound of the present invention comprises a light chain variable domain and a heavy chain variable domain, or the antigen binding fragments thereof, described in the preceding two paragraphs. For example, the binding compound may comprise a CDRL1 selected from the group consisting of SEQ ID NOs: 6 and 15; a CDRL2 selected from the group consisting of SEQ NOs: 7 and 21: a. CDRL3 consisting of SEQ ID NO: 8; a CDRH1 selected from the group consisting of SEQ ID NOs: 3 and 20; a CDRH2 consisting of SEQ ID NO: 4; and a CDRH3 consisting of SEQ ID NO: 5.

In some embodiments, the binding compound comprises a framework region, wherein the amino acid sequence of the framework region is all or substantially all of a human immunoglobulin amino acid sequence.

In some embodiments the light chain and/or heavy chain variable domains comprise a variant of one or more of the CDRs. In various embodiments the variant domain comprises up to 1, 2, 3, 4, 5, 6, 7, 8, 9, 10 or more conservatively modified amino acid residues relative to the sequence of the respective SEQ ID NOs. Conservative amino acid substitutions are provided at Table 1.

In some embodiments the light chain variable domain comprises a sequence selected from the group consisting of SEQ ID NOs: 12, 14 and 23 or a variant thereof. In some embodiments the heavy chain variable domain comprises a sequence selected from the group consisting of SEQ ID NOs: 11, 13 and 22. In various embodiments the variant variable domain comprises up to 1, 2, 3, 4, 5, 6, 7, 8, 9, 10, 15, 20, 30, 40 or 50 or more conservatively modified amino acid residues relative to the sequence of the respective SEQ ID NOs. In yet a further embodiment, the binding compound comprises a light chain variable domain and a heavy chain variable domain, or the antigen binding fragments thereof, described in this paragraph.

In other embodiments the binding compound of the present invention comprises a light chain variable domain, or an antigen binding fragment thereof, consisting essentially of a sequence selected from the group consisting of SEQ ID NOs: 12, 14 and 23, and/or a heavy chain variable domain, or an antigen binding fragment thereof, consisting essentially of a sequence selected from the group consisting of SEQ ID NOs: 11, 13 and 22.

In other embodiments the binding compound of the present invention comprises a light chain variable domain, or an antigen binding fragment thereof, having at least 50%, 75%, 80%, 85%, 90%, 95%, 98% or 99% sequence homology with a sequence selected from the group consisting of SEQ ID NOs: 12, 14 and 23, and/or a heavy chain variable domain, or an antigen. binding fragment thereof, having at least 50%, 75%, 80%, 85%, 90%, 95%, 98% or 99% sequence homology with a sequence selected from the group consisting of SEQ ID NOs: 11, 13 and 22.

In another embodiment the binding compound of the present invention (hu8B10-b) comprises the antibody light chain variable domain of SEQ ID NO: 14 and the antibody heavy chain variable domain of SEQ ID NO: 13, or an antigen binding fragment thereof.

In other embodiments the binding compound of the present invention comprises a light chain variable domain, or an antigen binding fragment thereof, comprising SEQ ID NO: 14 with one or more of the following substitutions: D56S and D56E, and/or a heavy chain variable domain, or an antigen binding fragment thereof, comprising SEQ ID NO: 13 with one or more of the following substitutions: N30S, N30D, N52S, N57S and N57D. In one embodiment the binding compound of the present invention (hu8B10-c) comprises the antibody light chain variable domain of SEQ ID NO: 23 and the antibody heavy chain variable domain of SEQ ID NO: 22, or an antigen binding fragment thereof

In some embodiments, the binding compound of the present invention comprises a full-length light chain polypeptide sequence selected from SEQ ID NOs: 17 and 25 and a full-length heavy chain polypeptide sequence selected from SEQ ID NOs: 16, 24 and 26. In specific embodiments, the binding compound comprises anti-IL-23R antibodies combining polypeptide chains having the sequences of SEQ ID NOs: 16 and 17 (hu8B10-b, IgG1), SEQ ID NOs: 24 and 25 (hu8B10-c, IgG1), or SEQ ID NOs: 25 and 26 (hu8B10-c, IgG4).

In one embodiment, the invention relates to antibodies that are able to block the binding of a binding compound of the present invention to human IL-23R in a cross-blocking assay. In various embodiments the antibody is able to block binding of human IL-23R to an antibody comprising the CDR sequences of antibody hu8B10-b or hu8B10-c, as disclosed herein. In another embodiment, the invention relates to binding compounds that are able to block IL-23R-mediated activity, such activities including but not limited to, binding IL-23, or mediating the proliferation or survival of Th17 cells.

In some embodiments, the binding compound of the present invention comprises a humanized antibody comprising the CDRs, or variants thereof, selected from the CDRs of the antibodies disclosed herein, in combination with human germline light chain and heavy chain variable domain framework sequences in place of the rodent frameworks of the parental antibodies.

In some embodiments, the binding compound of the present invention further comprises a heavy chain constant region, wherein the heavy chain constant region comprises or aγ1, γ2, γ3, or γ4 human heavy chain constant region or a variant thereof. In various embodiments the light chain constant region comprises a lambda or a kappa human light chain constant region.

In various embodiments the binding compounds of the present invention are polyclonal, monoclonal, chimeric, humanized or fully human antibodies or fragments thereof. The present invention also contemplates that the antigen binding fragment is an antibody fragment selected from the group consisting of Fab, Fab′, Fab′-SH, Fv, scFv, F(ab′)₂, and a diabody.

The present invention encompasses a method of suppressing an immune response in a human subject comprising administering to a subject in need thereof an antibody (or a antigen binding fragment thereof) specific for IL-23R in an amount effective o block IL-23 signaling. In some embodiments, the antibody specific for IL-23R is the humanized or chimeric antibody. In further embodiments, the immune response is an inflammatory response including arthritis, psoriasis, and inflammatory bowel disease. In other embodiments, the immune response is an autoimmune response, including multiple sclerosis, uveitis, systemic lupus erythematosus and diabetes. In another embodiment, the subject has cancer and the immune response is a Th17 response.

The present invention also contemplates administering an additional immunosuppressive or anti-inflammatory agent. The binding compounds of the present invention can be in a pharmaceutical composition comprising the binding compound, or antigen binding fragment thereof in combination with a pharmaceutically acceptable carrier or diluent. In a further embodiment, the pharmaceutical composition further comprises an immunosuppressive or anti-inflammatory agent.

The present invention encompasses an isolated nucleic acid encoding the polypeptide sequence of an antibody embodiment of the binding compound of the present invention. The nucleic acid can be in an expression vector operably linked to control sequences recognized by a host cell transfected with the vector. Also encompassed is a host cell comprising the vector, and a method of producing a polypeptide comprising culturing the host cell under conditions wherein the nucleic acid sequence is expressed, thereby producing the polypeptide, and recovering the polypeptide from the host cell or medium. In one embodiment, the nucleic acid sequence of the heavy chain consists of SEQ ID NO: 27 and the nucleic acid sequence of the light chain consists of SEQ ID NO; 28. In another embodiment, the nucleic acid sequence of the heavy chain comprises residues 58-1395 of SEQ ID NO: 27 and the nucleic acid sequence of the light chain comprises residues 58-699 of SEQ ID NO: 28. In another embodiment, the invention encompasses expression vectors comprising one or more of the nucleic acid sequences of the invention, e.g. the sequence of a heavy chain, a light chain, or both heavy and light chains. In yet another embodiment, the invention encompasses a host cell containing at least one of the expression vectors of the invention. e.g. a host cell comprising a single expression vector encoding both the heavy and the light chains of an antibody of the present invention, or comprising separate expression vectors encoding the heavy and the light chains.

In various embodiments, the invention relates to use of a binding compound of the present invention in the manufacture of medicaments for the treatment of disorders including, but not limited to, inflammatory disease, autoimmune disease, cancer, infectious disease (e.g. bacterial, mycobacterial, viral or fungal infection, including chronic infections), arthritis, psoriasis, inflammatory bowel disease, multiple sclerosis, uveitis, systemic lupus erythematosus and diabetes. In other embodiments the invention relates to compositions limited by the aforementioned uses.

In other embodiments the invention relates to pharmaceutical compositions comprising a binding compound of the present invention for treating disorders including, but not limited to, inflammatory disease, autoimmune disease, cancer, infectious disease (e.g. bacterial, mycobacterial, viral or fungal infection, including chronic infections), arthritis, psoriasis, inflammatory bowel disease, multiple sclerosis, uveitis, systemic lupus erythematosus and diabetes.

In some embodiments, the binding compound or pharmaceutical composition of the present invention induces a prolonged period of remission from disease symptoms in a subject, such that the dosing interval can be extended to much longer than the half-life of the binding compound in the subject, for example in the treatment of a relapsing-remitting disease. In various embodiments, the interval between one administration and another is 6-, 8-, 10-. 12-, 16-, 20-, 24-, 30-weeks or longer. In other embodiments a single administration is sufficient to permanently prevent relapses.

### BRIEF DESCRIPTION OF THE DRAWINGS

FIG. 1 shows comparisons of rat (“r”) and humanized (“hu”) anti-human IL-23R. antibody clone heavy chain variable domain sequences. Sequences are provided for clones r8B10, hu8B10, hu8B10-b and hu8B10-c. CDRs are indicated. Hu8B10-b and hu8B10-c residues that differ from the corresponding hu8B10 residues are underlined. Cross references to sequence identifiers are provided. See Table 4 and the Sequence Listing. Antibodies r8B10 and hu8B10 are also disclosed in co-pending, commonly assigned Int'l Pat. App. No. PCT/US2008/002534, published as WO 2008/106134 on Sept. 4, 2008.

FIG. 2 shows comparisons of rat and humanized anti-human IL-23R antibody clone light chain variable domain sequences. Sequences are provided for clones r8B10, hu8B10, hu8B10-b and hu8B10-c. CDRs are indicated. Hu8B10-b and hu8B10-c residues that differ from the corresponding hu8B10 residues are underlined. Cross references to sequence identifiers are provided. See Table 4 and the Sequence Listing. Antibodies r8B10 and hu8B10 are also disclosed in co-pending, commonly assigned Int'l Pat. App. No. PCT/US2008/002534, published as WO 2008/106134 on Sep. 4, 2008.

### DETAILED DESCRIPTION

As used herein, including the appended claims, the singular forms of words such as “a,” “an,” and “the,” include their corresponding plural references unless the context clearly dictates otherwise. Table 4 below provides a listing of sequence identifiers used in this application.

All references cited herein are incorporated by reference to the same extent as if each individual publication, database entry (e.g. Genbank sequences or GeneID entries), patent application, or patent, was specifically and individually indicated to be incorporated by reference. This statement of incorporation by reference is intended by Applicants, pursuant to 37 C.F.R, §1.57(b)(1), to relate to each and every individual publication, database entry (e.g. Genbank sequences or GeneID entries), patent application, or patent, each of which is clearly identified in compliance with 37 C.F.R. §1.57(b)(2). even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

### I. Definitions

“Proliferative activity” encompasses an activity that promotes, that is necessary for, or that is specifically associated with, e.g., normal cell division, as well as cancer, tumors, dysplasia, cell transformation, metastasis, and angiogenesis.

“Administration” and “treatment,” as it applies to an animal, human, experimental subject, cell, tissue, organ, or biological fluid, refers to contact of an exogenous pharmaceutical, therapeutic, diagnostic agent, or composition to the animal, human, subject, cell, tissue, organ, or biological fluid. “Administration” and “treatment” can refer, e.g., to therapeutic, pharmacokinetic, diagnostic, research, and experimental methods. Treatment of a cell encompasses contact of a reagent to the cell, as well as contact of a reagent to a fluid, where the fluid is in contact with the cell. “Administration” and “treatment” also means in vitro and ex vivo treatments, e.g., of a cell, by a reagent, diagnostic, binding composition, or by another cell. “Treatment,” as it applies to a human, veterinary, or research subject, refers to therapeutic treatment, prophylactic or preventative measures, to research and diagnostic applications. “Treatment” as it applies to a human, veterinary, or research subject, or cell, tissue, or organ, encompasses contact of an agent with animal subject, a cell, tissue, physiological compartment, or physiological fluid. “Treatment of a cell” also encompasses situations where the agent contacts IL-23 receptor (IL-23R/IL-12Rβ1 heterodimer), e.g., in the fluid phase or colloidal phase, but also situations where the agonist or antagonist does not contact the cell or the receptor.

As used herein, “inhibit” or “treat” or “treatment” includes a postponement of development of the symptoms associated with autoimmune disease or pathogen-induced immunopathology and/or a reduction in the severity of such symptoms that will or are expected to develop. The terms further include ameliorating existing uncontrolled or unwanted autoimmune-related or pathogen-induced immunopathology symptoms, preventing additional symptoms, and ameliorating or preventing the underlying causes of such symptoms. Thus, the terms denote that a beneficial result has been conferred on a vertebrate subject with an autoimmune or pathogen-induced immunopathology disease or symptom, or with the potential to develop such a disease or symptom.

As used herein, the term “therapeutically effective amount” or “effective amount” refers to an amount of an IL-23R-specific binding compound, e.g. and antibody, that when administered alone or in combination with an additional therapeutic agent to a cell, tissue, or subject is efthctive to prevent or ameliorate the autoimmune disease or pathogen-induced immunopathology associated disease or condition or the progression of the disease. A therapeutically effective dose further refers to that amount of the compound sufficient to result in amelioration of symptoms, e.g., treatment, healing, prevention or amelioration of the relevant medical condition, or an increase in rate of treatment, healing, prevention or amelioration of such conditions. When applied to an individual active ingredient administered alone, a therapeutically effective dose refers to that ingredient alone. When applied to a combination, a therapeutically effective dose refers to combined amounts of the active ingredients that result in the therapeutic effect, whether administered in combination, serially or simultaneously. An effective amount of therapeutic will decrease the symptoms typically by at least 10%; usually by at least 20%; preferably at least about 30%; more preferably at least 40%, and most preferably by at least 50%.

As used herein, the term “antibody” refers to any form of antibody that exhibits the desired biological activity. Thus, it is used in the broadest sense and specifically covers monoclonal antibodies (including full length monoclonal antibodies), polyclonal antibodies, multispecific antibodies (e.g., bispecific antibodies), chimeric antibodies, humanized antibodies, fully human antibodies, etc. so long as they exhibit the desired biological activity.

As used herein, the terms “IL-23R binding fragment,” “binding fragment thereof” or “antigen binding fragment thereof” encompass a fragment or a derivative of an antibody that still substantially retains its biological activity of binding to IL-23R. In some embodiments, an antibody or antigen binding fragment thereof of the present invention inhibits IL-23 signaling via the IL-23 receptor, such inhibition being referred to herein as “IL-23R inhibitory activity.” Because antagonists of IL-23R will have the biological activity of inhibiting IL-23 signaling, such antagonists are said (interchangeably) to inhibit IL-23R, inhibit IL-23, or inhibit both IL-23/IL-23R. The term “antibody fragment” or IL-23R binding fragment refers to a portion of a full length antibody, generally the antigen binding or variable region thereof. Examples of antibody fragments include Fab, Fab′, F(ab′)₂, and Fv fragments; diabodies; linear antibodies; single-chain antibody molecules, e.g., sc-Fv; and multispecific antibodies formed from antibody fragments. Typically, a binding fragment or derivative retains at least 10% of its IL-23R inhibitory activity. Preferably, a binding fragment or derivative retains at least 25%, 50%, 60%. 70%, 80%, 90%, 95%, 99% or 100% (or more) of its IL-23R inhibitory activity, although any binding fragment with sufficient affinity to exert the desired biological effect will be useful. It is also intended that, when specified, a IL-23R binding fragment can include variants having conservative amino acid substitutions that do not substantially alter its biologic activity.

As used herein, unless otherwise indicated, an antibody or antigen binding fragment thereof that is said to comprise a polypeptide (such as a light or heavy chain) is intended to also encompass antibodies or fragments that comprise multiple copies of the polypeptide. Specifically, antibodies said to comprise “a” heavy and/or “a” light chain encompass, but are not limited to, natural antibodies structures that consist of two copies of the light chain and two copies of the heavy chain.

The term “monoclonal antibody”, as used herein, refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic epitope. In contrast, conventional (polyclonal) antibody preparations typically include a multitude of antibodies directed against (or specific for) different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al. (1975) Nature 256: 495, or may he made by recombinant DNA methods (see, e.g., U.S. Pat. No. 4,816,567). The “monoclonal antibodies” may also be isolated from phage antibody libraries using the techniques described in Clackson et al. (1991) Nature 352: 624-628 and Marks et al. (1991) J. Mol. Biol. 222: 581-597, for example.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity, U.S. Pat. No. 4,816,567; Morrison et al (1984) Proc. Natl. Acad. Sci. USA 81: 6851-6855.

A “domain antibody” is an immunologically functional immunoglobulin fragment containing only the variable region of a heavy chain or the variable region of a light chain. In some instances, two or more V_{H} regions are covalently joined with a peptide linker to create a bivalent domain antibody. The two V_{H} regions of a bivalent domain antibody may target the same or different antigens.

A “bivalent antibody” comprises two antigen binding sites. In some instances, the two binding sites have the same antigen specificities. However, bivalent antibodies may be bispecific. As used herein, the term “bispecific antibody” refers to an antibody, typically a monoclonal antibody, having binding specificities for at least two different antigenic epitopes. In one embodiment, the epitopes are from the same antigen. In another embodiment, the epitopes are from two different antigens. Methods for making bispecific antibodies are known in the art. For example, bispecific antibodies can be produced recombinantly using the co-expression of two immunoglobulin heavy chain/light chain pairs. See, e.g., Milstein et al. (1983) Nature 305: 537-39. Alternatively, bispecific antibodies can be prepared using chemical linkage. See, e.g., Brennan et al. (1985) Science 229:81. Bispecific antibodies include bispecific antibody fragments. See, e.g., Holliger et al. (1993) Proc. Natl. Acad. Sci. U.S.A. 90:6444-48, Gruber et al. (1994) J. Immunol. 152:5368.

As used herein, the term “single-chain Fv” or “scFv” antibody refers to antibody fragments comprising the V_{H} and V_{L} domains of antibody, wherein these domains are present in a single polypeptide chain. Generally, the Fv polypeptide further comprises a polypeptide linker between the V_{H} and V_{L} domains which enables the scFv to form the desired structure for antigen binding. For a review of scFv, see Pluckthun (1994) THE PHARMACOLOGY OF MONOCLONAL ANTIBODIES, vol. 113, Rosenburg and Moore eds. Springer-Verlag, New York, pp. 269-315.

The monoclonal antibodies herein also include carnelized single domain antibodies. See, e.g., Muyldermans et al. (2001) Trends Biocheni. Sci. 26:230; Reichmann et al. (1999) J. Immunol. Methods 231:25; WO 94/04678; WO 94/25591.; U.S. Pat. No. 6,005,079). in one embodiment, the present invention provides single domain antibodies comprising two V_{H} domains with modifications such that single domain antibodies are formed.

As used herein, the term “diabodies” refers to small antibody fragments with two antigen-binding sites, which fragments comprise a heavy chain variable domain (V_{H}) connected to a light chain variable domain (V_{L}) in the same polypeptide chain (V_{H}-V_{L} or V_{L}-V_{H}). By using a linker that is too short to allow pairing between the two domains on the same chain, the domains are forced to pair with the complementary domains of another chain and create two antigen-binding sites. Diabodies are described more fully in, e.g., EP 404,097: WO 93/11 161; and Holliger et al. (1993) Proc. Natl. Acad. Sci. USA 90: 6444-6448. For a review of engineered antibody variants generally see Holliger and Hudson (2005) Nat. Biotechnol. 23:1126-1136.

As used herein, the term “humanized antibody” refers to forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The prefix “hum”, “hu” or “h” is added to antibody clone designations when necessary to distinguish humanized antibodies from parental rodent antibodies (although these same designations, depending on the context, may also indicate the human form of a particular protein). The humanized forms of rodent antibodies will generally comprise the same CDR sequences of the parental rodent antibodies, although certain amino acid substitutions may be included to increase affinity, increase stability of the humanized antibody, or for other reasons.

The antibodies of the present invention also include antibodies with modified (or blocked) Fc regions to provide altered effector functions. See, e.g., U.S. Pat. No. 5,624,821; WO 2003/086310; WO 2005/120571; WO 2006/0057702; Presta (2006) Adv. Drug Delivery Rev. 58:640-656. Such modification can be used to enhance or suppress various reactions of the immune system, with possible beneficial effects in diagnosis and therapy. Alterations of the Fc region include amino acid changes (substitutions, deletions and insertions), glycosylation or deglycosylation, and adding multiple Fc. Changes to the Fc can also alter the half-life of antibodies in therapeutic antibodies. A longer half-life may result in less frequent dosing, with the concomitant increased convenience and decreased use of material. See Presta (2005) J. Allergy Clin. Immunol. 116:731 at 734-35.

The antibodies of the present invention also include antibodies with intact Fc regions that provide full effector functions, e.g. antibodies of isotype IgGl, which induce complement-dependent cytotoxicity (CDC) or antibody dependent cellular cytotoxicity (ADCC) in the a targeted cell. In some embodiments, the antibodies of the present invention are administered to selectively deplete IL-23R-positive cells from a population of cells. In one embodiment, this depletion of IL-23R-positive cells is the depletion of pathogenic Th17 cells. Depletion of such pathogenic T cell subset may result in sustained remission when effected in subjects suffering from a relapsing/remitting autoimmune disease.

The antibodies of the present invention also include antibodies conjugated to cytotoxic payloads, such as cytotoxic agents or radionuclides. Such antibody conjugates may be used in immunotherapy to selectively target and kill cells expressing IL-23R on their surface. Exemplary cytotoxic agents include ricin, vinca alkaloid, methotrexate, Psuedomonas exotoxin, saporin, diphtheria toxin, cisplatin, doxorubicin, abrin toxin, gelonin and pokeweed antiviral protein. Exemplary radionuclides for use in immunotherapy with the antibodies of the present invention include ¹²⁵I, ¹³¹I, ⁹⁰Y, ⁶⁷Cu, ²¹¹At, ¹⁷⁷Lu, ¹⁴³Pr and ²¹³Bi. See, e.g., U.S. Patent Application Publication No. 2006/0014225.

The term “fully human antibody” refers to an antibody that comprises human immunoglobulin protein sequences only. A fully human antibody may contain murine carbohydrate chains if produced in a mouse, in a mouse cell, or in a hybridoma derived from a mouse cell. Similarly, “mouse antibody” or “rat antibody” refer to an antibody that comprises only mouse or rat immunoglobulin sequences, respectively. A fully human antibody may be generated in a human being, in a transgenic animal having human immunoglobulin germline sequences, by phage display or other molecular biological methods.

As used herein, the term “hypervariable region” refers to the amino acid residues of an antibody that are responsible for antigen-binding. The hypervariable region comprises amino acid residues from a “complementarily determining region” or “CDR” (e.g. residues 24-34 (CDRL1), 50-56 (CDRL2) and 89-97 (CDRL3) in the light chain variable domain and residues 31-35 (CDRH1), 50-65 (CDRH2) and 95-102 (CDRH3) in the heavy chain variable domain (Kabat et al. (1991) Sequences of Proteins of Immunological Interest, 5th Ed. Public Health Service, National Institutes of Health, Bethesda, Md.) and/or those residues from a “hypervariable loop” (i.e. residues 26-32 (L1), 50-52 (L2) and 91-96 (L3) in the light chain variable domain and 26-32 (H1), 53-55 (H2) and 96-101 (H3) in the heavy chain variable domain (Chothia and Lesk (1987) J. Mol. Biol. 196: 901-917). As used herein, the term “framework” or “FR” residues refers to those variable domain residues other than the hvpervariable region residues defined herein as CDR residues. The residue numbering above relates to the Kabat numbering system and does not necessarily correspond in detail to the sequence numbering in the accompanying Sequence Listing.

Sequence variants in both the CDR and framework regions are contemplated, and may be represented as “XbbZ”, in which amino acid “X” at position “bb” is replaced by amino acid “Z”, and wherein X and Z are in either triple- or single-letter amino acid code, and position number “bb” is typically defined with respect to the numbering of a specific sequence disclosed in the Sequence Listing. In cases where a residue is deleted from the sequence, “Z” is replaced by “del”. In the interests of clarity, a sequence said to be “comprising,” “consisting of” or “consisting essentially of” a recited sequence (e.g. SEQ ID NO: Y) having an “XbbZ” alteration, or being an “XbbZ variant” of the recited sequence, is intended to refer to an altered version of the recited sequence in which residue X is replaced by residue Z, such that the resulting final sequence comprises Z at position bb and not X, as is common usage in the art, regardless of how the phrases “comprising”, “consisting of” or “consisting essentially of” might otherwise be construed under the patent law.

“Binding compound” refers to a molecule, small molecule, macromolecule, polypeptide, antibody or fragment or analogue thereof, or soluble receptor, capable of binding to a target. “Binding compound” also may refer to a complex of molecules, e.g., a non-covalent complex, to an ionized molecule, and to a covalently or non-covalently modified molecule, e.g., modified by phosphorylation, acylation, cross-linking, cyclization, or limited cleavage, that is capable of binding to a target. When used with reference to antibodies, the term “binding compound” refers to both antibodies and antigen binding fragments thereof. “Binding” refers to an association of the binding compound with a target where the association results in reduction in the normal Brownian motion of the binding compound, in cases where the binding compound can be dissolved or suspended in solution. “Binding composition” refers to a molecule, e.g. a binding compound, in combination with a stabilizer, excipient, salt, buffer, solvent, or additive, capable of binding to a target.

“Conservatively modified variants” or “conservative substitution” refers to substitutions of amino acids are known to those of skill in this art and may often be made even in essential regions of the polypeptide without altering the biological activity of the resulting molecule. Such exemplary substitutions are preferably made in accordance with those set forth in Table 1 as follows:

TABLE 1
Exemplary Conservative Amino Acid Substitutions

| Original residue | Conservative substitution |
|---|---|
| Ala (A) | Gly; Ser |
| Arg (R) | Lys; His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala |
| Ser (S) | Thr |
| Thr (T) | Ser |
| Trp (W) | Tyr; Phe |
| Tyr (Y) | Trp; Phe |
| Val (V) | Ile; Leu |

Those of skill in this art recognize that, in general, single amino acid substitutions in non-essential regions of a polypeptide may not substantially alter biological activity. See, e.g., Watson et al, (1987) Molecular Biology of the Gene, The Benjamin/Cummings Pub. Co., p. 224 (4th Edition).

The phrase “consists essentially of,” or variations such as “consist essentially of” or “consisting essentially of,” as used throughout the specification and claims, indicate the inclusion of any recited elements or group of elements, and the optional inclusion of other elements, of similar or different nature than the recited elements, that do not materially change the basic or novel properties of the specified dosage regimen, method, or composition. As a non-limiting example, a binding compound that consists essentially of a recited amino acid sequence may also include one or more amino acids, including substitutions of one or more amino acid residues, that do not materially affect the properties of the binding compound.

“Effective amount” encompasses an amount sufficient to ameliorate or prevent a symptom or sign of the medical condition. Such an effective amount need not necessarily completely ameliorate or prevent such symptom or sign. Effective amount also means an amount sufficient to allow or facilitate diagnosis. An effective amount for a particular patient or veterinary subject may vary depending on factors such as the condition being treated, the overall health of the patient, the method route and dose of administration and the severity of side affects. See, e.g., U.S. Pat. No. 5,888,530. An effective amount can be the maximal dose or dosing protocol that avoids significant side effects or toxic effects. The effect will result in an improvement of a diagnostic measure or parameter by at least 5%, usually by at least 10%, more usually at least 20%, most usually at least 30%, preferably at least 40%, more preferably at least 50%, most preferably at least 60%, ideally at least 70%, more ideally at least 80%, and most ideally at least 90%, where 100% is defined as the diagnostic parameter shown by a normal subject. See, e.g., Maynard et al. (1996) A Handbook of SOPs for Good Clinical Practice, Interpharm Press, Boca Raton, Fla.; Dent (2001) Good Laboratory and Good Clinical Practice, Urch Publ., London, UK.

“Immune condition” or “immune disorder” encompasses, e.g., pathological inflammation, an inflammatory disorder, and an autoimmune disorder or disease. “Immune condition” also refers to infections, persistent infections, and proliferative conditions, such as cancer, tumors, and angiogenesis, including infections, tumors, and cancers that resist eradication by the immune system. “Cancerous condition” includes, e.g., cancer, cancer cells, tumors, angiogenesis, and precancerous conditions such as dysplasia.

“Inflammatory disorder” means a disorder or pathological condition where the pathology results, in whole or in part, from, e.g., a change in number, change in rate of migration, or change in activation, of cells of the immune system. Cells of the immune system include, e.g., T cells, B cells, monocytes or macrophages, antigen presenting cells (APCs), dendritic cells, microglia, NK cells, NKT cells, neutrophils, eosinophils, mast cells, or any other cell specifically associated with the immunology, for example, cytokine-producing endothelial or epithelial cells.

An “IL-17-producing cell” means a T cell that is not a classical TH1-type T cell or classical TH2-type T cell, referred to as Th17 cells. Th17 cells are discussed in greater detail at Cua and Kastelein (2006) Nat. Immunol. 7:557-559; Tato and O'Shea (2006) Nature 441:166-168; Iwakura and lshigame (2006) J. Clin. Invest. 116:1218-1222. “IL-17-producing cell” also means a T cell that expresses a gene or polypeptide of Table 10B of U.S. Patent Application Publication No. 2004/0219150 (e.g., mitogen responsive P-protein; chemokine ligand 2; interleukin-17 (IL-17); transcription factor RAR related; and/or suppressor of cytokine signaling 3), where expression with treatment by an IL-23 agonist is greater than treatment with an IL-12 agonist, where “greater than” is defined as follows. Expression with an IL-23 agonist is ordinarily at least 5-fold greater, typically at least 10-fold greater, more typically at least 15-fold greater, most typically at least 20-fold greater, preferably at least 25-fold greater, and most preferably at least 30-fold greater, than with IL-12 treatment. Expression can be measured, e.g., with treatment of a population of substantially pure IL-17 producing cells. A Th17 response is an immune response in which the activity and/or proliferation of Th17 cells are enhanced, typically coupled with a repressed Th1 response.

Moreover, “IL-17-producing cell” includes a progenitor or precursor cell that is committed, in a pathway of cell development or cell differentiation, to differentiating into an IL-17-producing cell, as defined above. A progenitor or precursor cell to the IL-17 producing cell can be found in a draining lymph node (DLN). Additionally, “IL-17-producing cell” encompasses an IL-17-producing cell, as defined above, that has been, e.g., activated, e.g., by a phorbol ester, ionophore, and/or carcinogen, further differentiated, stored, frozen, desiccated, inactivated, partially degraded, e.g., by apoptosis, proteolysis, or lipid oxidation, or modified, e.g., by recombinant technology.

As used herein, the term “isolated nucleic acid molecule” refers to a nucleic acid molecule that is identified and separated from at least one contaminant nucleic acid molecule with which it is ordinarily associated in the natural source of the antibody nucleic acid. An isolated nucleic acid molecule is other than in the form or setting in which it is found in nature. Isolated nucleic acid molecules therefore are distinguished from the nucleic acid molecule as it exists in natural cells. However, an isolated nucleic acid molecule includes a nucleic acid molecule contained in cells that ordinarily express the antibody where, for example, the nucleic acid molecule is in a chromosomal location different from that of natural cells.

The expression “control sequences” refers to DNA sequences involved in the expression of an operably linked coding sequence in a particular host organism. The control sequences that are suitable for prokaryotes, for example, include a promoter, optionally an operator sequence, and a ribosome binding site. Eukaryotic cells are known to use promoters, polyadenylation signals, and enhancers.

A nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading frame. However, enhancers do not have to be contiguous. Linking is accomplished by ligation at convenient restriction sites. If such sites do not exist, the synthetic oligonucleotide adaptors or linkers are used in accordance with conventional practice.

As used herein, the expressions “cell,” “cell line,” and “cell culture” are used interchangeably and all such designations include progeny. Thus, the words “transformants” and “transformed cells” include the primary subject cell and cultures derived therefrom without regard for the number of transfers. It is also understood that all progeny may not be precisely identical in DNA content, due to deliberate or inadvertent mutations. Mutant progeny that have the same function or biological activity as screened for in the originally transformed cell are included. Where distinct designations are intended, it will be clear from the context.

As used herein, “polymerase chain reaction” or “PCR” refers to a procedure or technique in which minute amounts of a specific piece of nucleic acid, RNA and/or DNA, are amplified as described in, e.g., U.S. Pat. No. 4,683,195. Generally, sequence information from the ends of the region of interest or beyond needs to be available, such that oligonucleotide primers can be designed; these primers be identical or similar in sequence to opposite strands of the template to be amplified. The 5′ terminal nucleotides of the two primers can coincide with the ends of the amplified material. PCR can be used to amplify specific RNA sequences, specific DNA sequences from total genomic DNA, and cDNA transcribed from total cellular RNA, bacteriophage or plasmid sequences, etc. See generally Mullis et al, (1987) Cold Spring Harbor Symp. Quant. Biol. 51:263; Erlich, ed., (1989) PCR TECHNOLOGY (Stockton Press, N.Y.) As used herein. PCR is considered to he one, but not the only, example of a nucleic acid polymerase reaction method for amplifying a nucleic acid test sample comprising the use of a known nucleic acid as a primer and a nucleic acid polymerase to amplify or generate a specific piece of nucleic acid.

As used herein, the term “germline sequence” refers to a sequence of unrearranged immunoglobulin DNA sequences, including rodent (e.g. mouse) and human germline sequences. Any suitable source of unrearranged immunoglobulin DNA may be used. Human germline sequences may be obtained, for example, from JOINSOLVER® germline databases on the website for the National Institute of Arthritis and Musculoskeletal and Skin Diseases of the United States National Institutes of Health. Mouse germline sequences may be obtained, for example, as described in Giudicelli et al. (2005) Nucleic Acids Res. 33:D256-D261.

To examine the extent of inhibition of IL-23/IL-23R activity, for example, samples or assays comprising a given, e.g., protein, gene, cell, or organism, are treated with a potential activating or inhibiting agent and are compared to control samples without the agent. Control samples, i.e., not treated with agent, are assigned a relative activity value of 100%. Inhibition is achieved when the activity value relative to the control is about 90% or less, typically 85% or less, more typically 80% or less, most typically 75% or less, generally 70% or less, more generally 65% or less, most generally 60% or less, typically 55% or less, usually 50% or less, more usually 45% or less, most usually 40% or less, preferably 35% or less, more preferably 30% or less, still more preferably 25% or less, and most preferably less than 20%. Activation is achieved when the activity value relative to the control is about 110%, generally at least 120%, more generally at least 140%, more generally at least 160%, often at least 180%, more often at least 2-fold, most often at least 2.5-fold, usually at least 5-fold, more usually at least 10-fold, preferably at least 20-fold, more preferably at least 40-fold, and most preferably over 40-fold higher.

Endpoints in activation or inhibition can be monitored as follows. Activation, inhibition, and response to treatment, e.g., of a cell, physiological. fluid, tissue, organ, and animal or human subject, can be monitored by an endpoint. The endpoint may comprise a predetermined quantity or percentage of, e.g., an indicia of inflammation, oncogenicity, or cell degranulation or secretion, such as the release of a cytokine, toxic oxygen, or a protease. The endpoint may comprise, e.g., a predetermined quantity of ion flux or transport; cell migration; cell adhesion; cell proliferation; potential. for metastasis; cell differentiation; and change in phenotype, e.g., change in expression of gene relating to inflammation, apoptosis, transformation, cell cycle, or metastasis (see, e.g., Knight (2000) Ann. Clin. Lab. Sci. 30:145-158; Hood and Cheresh (2002) Nature Rev. Cancer 2:91-100; Timme et al. (2003) Curr. Drug Targets 4:251-261; Robbins and Itzkowitz (2002) Med. Clin. North Am. 86:1467-1495; Grady and Markowitz (2002) Annu. Rev. Genomics Hum. Genet. 3:101-128; Bauer, et al. (2001) Glia 36:235-243; Stanimirovic and Satoh (2000) Brain Pathol. 10:113-126).

An endpoint of inhibition is generally 75% of the control or less, preferably 50% of the control or less, more preferably 25% of the control or less, and most preferably 10% of the control or less. Generally, an endpoint of activation is at least 150% the control, preferably at least two times the control, more preferably at least four times the control, and most preferably at least 10 times the control.

“Small molecule” is defined as a molecule with a molecular weight that is less than 10 kDa, typically less than 2 kDa, and preferably less than 1 kDa. Small molecules include, but are not limited to, inorganic molecules, organic molecules, organic molecules containing an inorganic component, molecules comprising a radioactive atom, synthetic molecules, peptide mimetics, and antibody mimetics. As a therapeutic, a small molecule may be more permeable to cells, less susceptible to degradation, and less apt to elicit an immune response than large molecules. Small molecules, such as peptide mimetics of antibodies and cytokines, as well as small molecule toxins are described. See, e.g., Casset et al. (2003) Biochem. Biophys. Res. Commun. 307:198-205; Muyldermans (2001) J. Biotechnol. 74:277-302; Li (2000) Nat. Biotechnol 18:1251-1256; Apostolopoulos et al. (2002) Curr. Med. Chem. 9:411-420; Monfardini et al. (2002) Curr. Pharm. Des. 8:2185-2199: Domingues et al. (1999) Nat. Struct. Biol. 6:652-656; Sato and Sone (2003) Biochem. J. 371:603-608; U.S. Pat. No. 6,326,482.

“Specifically” or “selectively” binds, when referring to a ligand/receptor, antibody/antigen, or other binding pair, indicates a binding reaction that is determinative of the presence of the protein in a heterogeneous population of proteins and other biologics. Thus, under designated conditions, a specified ligand binds to a particular receptor and does not bind in a significant amount to other proteins present in the sample. As used herein, an antibody is said to bind specifically to a polypeptide comprising a given sequence (in this case IL-23R) if it binds to polypeptides comprising the sequence of IL-23R but does not bind to proteins lacking the sequence of IL-23R. For example, an antibody that specifically binds to a polypeptide comprising IL-23R may bind to a FLAG®-tagged form of IL-23R but will not bind to other FLAG®-tagged proteins.

The antibody, or binding composition derived from the antigen-binding site of an antibody, of the contemplated method binds to its antigen with an affinity that is at least two fold greater, preferably at least ten times greater, more preferably at least 20-times greater, and most preferably at least 100-times greater than the affinity with unrelated antigens. In a preferred embodiment the antibody will have an affinity that is greater than about 10⁹ liters/mol, as determined, e.g., by Scatchard analysis. Munsen et al. (1980) Analyt. Biochem. 107:220-239.

As used herein, the term “immunomodulatory agent” refers to natural or synthetic agents that suppress or modulate an immune response. The immune response can be a humoral or cellular response. Immunomodulatory agents encompass immunosuppressive or anti-inflammatory agents.

“Immunosuppressive agents,” “immunosuppressive drugs,” or “immunosuppressants” as used herein are therapeutics that are used in immunosuppressive therapy to inhibit or prevent activity of the immune system. Clinically they are used to prevent the rejection of transplanted organs and tissues (e.g. bone marrow, heart, kidney, liver), and/or in the treatment of autoimmune diseases or diseases that are most likely of autoimmune origin (e.g. rheumatoid arthritis, myasthenia gravis, systemic lupus erythematosus, ulcerative colitis, multiple sclerosis). Immunosuppressive drugs can be classified into four groups: glucocorticoids cytostatics; antibodies (including Biological Response Modifiers or DMARDs); drugs acting on immunophilins; other drugs, including known chemotherapeutic agents used in the treatment of proliferative disorders. For multiple sclerosis, in particular, antibodies of the present inverntion can be administered in conjunction with a new class of myelin binding protein-like therapeutics, known as copaxones.

“Anti-inflammatory agents” or “anti-inflammatory drugs”, is used to represent both steroidal and non-steroidal therapeutics. Steroids, also known as corticosteroids, are drugs that closely resemble cortisol, a hormone produced naturally by adrenal glands. Steroids are used as the main treatment for certain inflammatory conditions, such as: Systemic vasculitis (inflammation of blood vessels); and Myositis (inflammation of muscle). Steroids might also be used selectively to treat inflammatory conditions such as: rheumatoid arthritis (chronic inflammatory arthritis occurring in joints on both sides of the body); systemic lupus erythematosus (a generalized disease caused by abnormal immune system function); Sjögren's syndrome (chronic disorder that causes dry eyes and a dry mouth).

Non-steroidal anti-inflammatory drugs, usually abbreviated to NSAIDs, are drugs with analgesic, antipyretic and anti-inflammatory effects—they reduce pain, fever and inflammation. The term “non-steroidal” is used to distinguish these drugs from steroids, which (amongst a broad range of other effects) have a similar eicosanoid-depressing, anti-inflammatory action. NSAIDs are generally indicated for the symptomatic relief of the following conditions: rheumatoid arthritis; osteoarthritis; inflammatory arthropathies (e.g. ankylosing spondylitis, psoriatic arthritis, Reiter's syndrome); acute gout; dysmenorrhoea; metastatic bone pain; headache and migraine; postoperative pain; mild-to-moderate pain due to inflammation and tissue injury; pyrexia; and renal colic. NSAIDs include salicylates, arlyalknoic acids, 2-arylpropionic acids (profens), N-arylanthranilic acids (fenamic acids), oxicams, coxibs, and sulphonanilides.

### II General

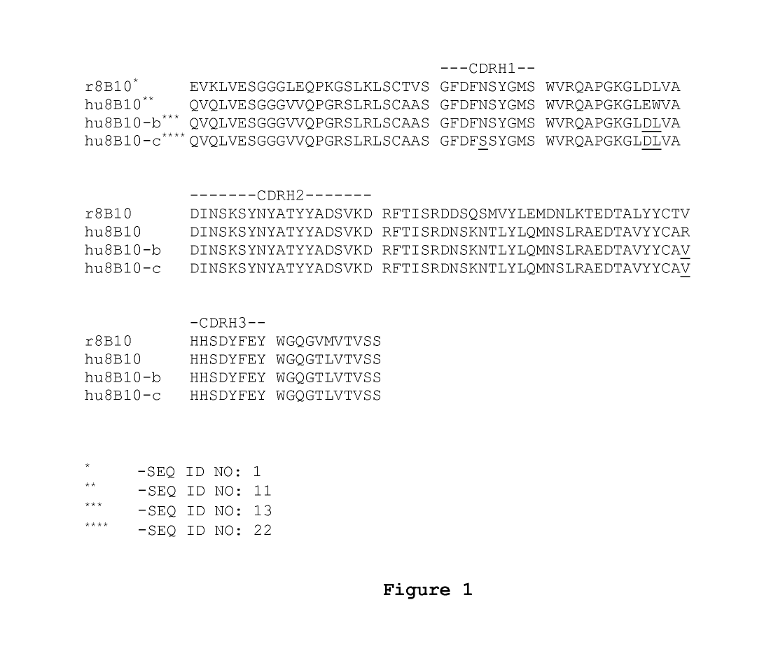

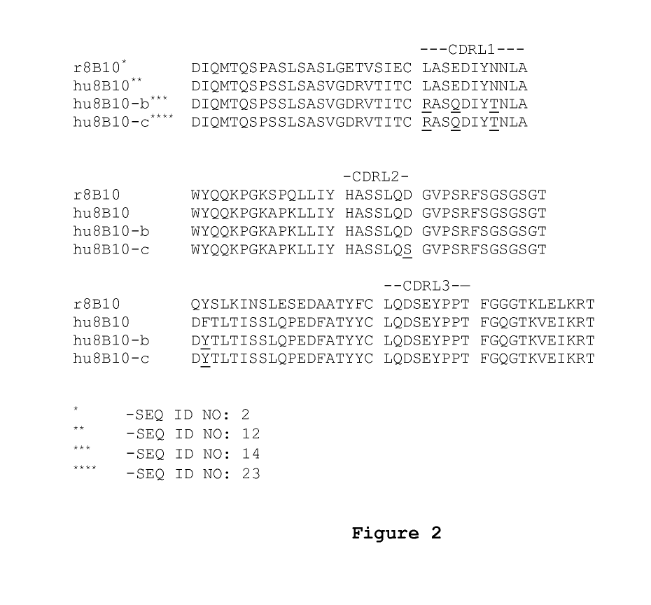

The present invention provides engineered anti-IL-23R antibodies and uses thereof to treat inflammatory, autoimmune, and proliferative disorders. The present invention is related to co-pending, commonly assigned Int'l Pat. App. No. PCT/US2008/002534, published as WO 2008/106134 on Sep. 4, 2008. The present application discloses additional sequences and data relating to humanized anti-human IL-23R antibody hu8B10-b and hu8B10-c and variants thereof, as described in greater detail infra at Examples 7 and 8, SEQ ID NOs: 13-25, and FIGS. 1 and 2.

A number of cytokines have a role in the pathology or repair of neurological disorders. IL-6, IL-17, interferon-gamma (IFNgamma, IFN-γ), and granulocyte colony-stimulating factor (GM-CSF) have been associated with multiple sclerosis. Matusevicius et al. (1999) Multiple Sclerosis 5:101-104; Lock et al. (2002) Nature Med. 8:500-508. IL-1α, IL-1β, and transforming growth factor-β 1 (TGF-β1) play a role in ALS, Parkinson's disease, and Alzheimer's disease. Hoozemans et al. (2001) Exp. Gerontal. 36:559-570; Griffin and Mrak (2002) J. Leukocyte Biol. 72:233-238; Ilzecka et al. (2002) Cytokine 20:239-243. TNF-α, IL-1β, IL-6, IL-8, interferon-gamma, and IL-17 appear to modulate response to brain ischemia. See, e.g., Kostulas et al. (1999) Stroke 30:2174-2179; Li et al. (2001) J. Neuroimmunol. 116:5-14. Vascular endothelial cell growth factor (VEGF) is associated with ALS. Cleveland and Rothstein (2001) Nature 2:806-819.

Inflammatory bowel disorders, e.g., Crohn's disease, ulcerative colitis, celiac disease, and irritable bowel syndrome, are mediated by cells of the immune system and by cytokines. For example, Crohn's disease is associated with increased IL-12 and IFNγ, while ulcerative colitis is associated with increased IL-5, IL-13, and transforming growth factor-beta (TGF-β). IL-17 expression may also increase in Crohn's disease and ulcerative colitis. See, e.g., Podolsky (2002) New Engl. J. Med. 347:417-429; Bouma and Strober (2003) Nat. Rev. Immunol. 3:521-533; Bhan et al. (1999) Immunol. Rev. 169:195-207; Hanauer (1996) New Engl. J. Med. 334:841-848; Green (2003) The Lancet 362:383-391; McManus (2003) New Engl. J. Med. 348:2573-2574; Horwitz and Fisher (2000) New Engl. J. Med. 344:1846-1850; Andoh et al. (2002) Int. J. Mol. Med. 10:631-634; Nielsen et al. (2003) Scand. J. Gastroentecol. 38:180-185; Fujino et al. (2003) Gut 52:65-70.

IL-23R has been implicated as a critical genetic factor in the inflammatory bowel disorders Crohn's disease and ulcerative colitis. Duerr et al. (2006) Science 314:1461. A genome-wide association study found that the gene for IL-23R was highly associated with Crohn's disease, with an uncommon coding variant (Arg381Gln) conferring strong protection against the disease. This genetic association confirms prior biological findings (Yen el al. (2006) J. Clin. Investigation 116:1218) suggesting that IL-23 and its receptor (including IL-23R) are promising targets for new therapeutic approached to treating IBD.

Inflammatory diseases of the skin, joints, CNS, as well as proliferative disorders elicit similar immune responses, thus IL-23/IL-23R blockade should provide inhibition of these immune mediated inflammatory disorders, without comprising the host ability to fight systemic infections. Antagonizing IL-23/IL-23R should relieve the inflammation associated with inflammatory bowel disease, Crohn's disease, ulcerative colitis, rheumatoid arthritis, psoriatic arthritis, psoriasis, ankylosing spondylitis and atopic dermatitis. Use of IL-23/IL-23R inhibitors will also provide inhibition of proliferative disorders, e.g., cancer and autoimmune disorders e.g., multiple sclerosis, type I diabetes, and SLE. Descriptions of IL-23 in these various disorders can be found in the following published PCT applications: WO 04/0811.90; WO 04/071517; WO 00/53631; and WO 01/18051. IL-23/IL-23R inhibitors may also find use in treatment of infections, including chronic infections, such as bacterial, mycobacterial, viral and fungal infections. See co-pending commonly assigned Int'l Pat. App. No. PCT/US2008/01 717, published as WO 2008/153610 on Dec. 18, 2008.

The cellular receptor complex required to transduce the IL-23 signal consists of two members of the tall signaling receptor subunits of the IL-6/IL-12 family of cytokines, the IL-23-specific IL-23R (see, e.g., Parham. et al. supra) and the IL-12Rβ1 subunit, which is shared with IL-12. The amino acid sequence for human IL-23R is found at GenBank Accession No: AAM44229 (SEQ ID NO: 9), and an mRNA sequence is found at GenBank Accession No: NM_{—}144701, which is hereby incorporated by reference in its entirety. The human IL-23R gene is described at NCBI GeneID No. 149233, which is hereby incorporated by reference in its entirety. Amino acid residues I-23 of human IL-23R represents the signal sequence, which is not present in mature IL-23R. The amino acid sequence for human IL-12Rβ1 is found at GenBank Accession No: NP_{—}005526, and provided at SEQ ID NO: 10. Amino acid residues 1-24 represent the signal sequence for human IL-12Rβ1.

Comparison of the natural roles of IL-12 and IL-23 suggest that targeting IL-23 for inhibition will cause fewer adverse side-effects when compared with inhibition of IL-12, or inhibition of both IL-23 and IL-12. Bowman et al. (2006) Curr. Opin. Infect. Dis. 19:245. While IL-12 is critical to mounting a systemic Th1-mediated immune responses, IL-23 (along with IL-6 and TNF-α) is thought to be responsible for promotion and maintenance of Th-17 cells. Such Th-17 cells are believed to be involved in responses to catastrophic injury, such as breach of the mucosal barrier of the lung or gut, and the resulting exposure to the deadly pathogens K. pneumoniae and C. rodentium. Such catastrophic injuries would almost certainly require an immediate immune response in the form of massive neutrophil influx. See Cua and Kastelein (2006) Nature Immunology 7:557. Because such catastrophic injuries and infections are relatively rare in modern society, and can be treated with antibiotics if they do occur, this Th-17 “nuclear option” may not be as critical to survival as it was earlier in human evolution. This suggests that disruption of IL-23/IL-23 receptor signaling may have a relatively minor side effect profile, since its natural activity is of little importance in modern society. See McKenzie. et al. (2006) Trends Immunol. 27:17.

The distinct subunit compositions of IL-12 receptor and IL-23 receptor make it possible to design therapy that targets only IL-23 receptor but not IL-12 receptor. Compounds that bind to and inhibit the activity of IL-23p19 or IL-23R, either in isolation of as components of their respective heterodimeric complexes, will inhibit IL-23 but not IL-12. There may also be compounds that are capable of binding to IL-12p40 when present in IL-23 but not in IL-12, or compounds that bind to and inhibit IL-12Rβ1 when present in the IL-23 receptor but not in IL-12 receptor. Such specific binding agents will also inhibit IL-23 activity but not IL-12 activity. IL-23/IL-23R specific agents would be expected to be safer (i.e. have a lower side effect profile) than agents that also inhibit IL-12.

Much of the early work on inhibition of IL-12 involved inhibition of IL-12p40. It has been subsequently realized that these experiments involved not only inhibition of IL-12 but also inhibition of IL-23, and that in fact the effects in many of these experiments were the result of inhibition of IL-23. Many disorders once thought to be caused by a pathogenic Th1 response, which could be ameliorated by inhibition of IL-12, have been shown instead to be caused by a Th17 response, which is ameliorated by inhibition of IL-23. Yen et al. (2006) J. Clin. Invest. 116:1310; Iwakura and Ishingame (2006) J. Clin. Invest. 116:1218.

IL-23 receptor, and specifically IL-23R, represents an attractive target for therapeutic intervention. IL-23R is expressed on the surface of Th17 cells, and the antibodies of the present invention may be used to deliver a cytotoxic payload specifically to that T-cell subset. If desired, bispecific reagents such as BITE antibodies may also be used to target a cytotoxic T cell response to IL-23R expressing Th17 cells. Bauerle et al. (2008) Drugs of the Future 33:137. Such cell-specific immunotherapy is particularly suited to treatment of disorders in which Th17 cells migrate from a tissue in which they are induced (e.g. the gut, lung or skin) into another tissue, such as CNS, since the cells could be targeted and killed prior to infiltrating the new target tissue and inducing disease (e.g. MS in the CNS). See Chen et al. (2006) J. Clin. Invest. 116:1317.

In other embodiments, however, it be preferable to avoid cytotoxic effects in cells expressing IL-23R, but instead to simply block IL-23 signaling. In the case of treatment of tumors (see, e.g., WO 2004/081190), for example, it may be preferable to block IL-23 signaling in Th0 cells, and thus block Th17 formation, without inducing cell killing. The Th0 cells will then be available for production cells of the Th1 lineage, induced by IL-12, which can then engage in productive immune surveillance of the tumor cells with beneficial effect. The compositions and methods of the present invention encompass both scenarios by providing binding compounds (antibodies) that may or may not enhance killing of IL-23R expressing cells, depending on which is the preferred approach for the specific therapeutic use. In some embodiments, antibodies or fragments thereof lacking effector function may be used, such as IgG antibodies other than subclass IgG₁, or Fab or other antibody fragments.

Targeting IL-23R has advantages over targeting IL-12Rβ1, the other subunit of the heterodimeric IL-23 receptor, since inhibition of IL-23R will disrupt IL-23 signaling but not disrupt IL-12 signaling. Inhibition of IL-12Rβ1 would disrupt signaling of both IL-23 and IL-12. In general principle a more narrowly targeted therapy is preferable, and in this specific case there are good reasons to expect that disruption of IL-12 would reduce the subject's ability to resist some infectious diseases and to maintain tumor surveillance.

Targeting IL-23R has advantages over targeting IL-23p19 despite the fact that the two therapeutic approaches both act by disrupting IL-23 signaling through its receptor. IL-23R is present at significantly lower levels than IL-23p19, and the amount of anti-IL-23R antibody necessary to block IL-23R would be expected to be correspondingly lower than would be required to block the activity of p19 in a subject. This reduction in the amount of antibody may provide several advantages, such as reducing the expense of treatment, decreasing the concentration and/or volume of the drug to be delivered, and facilitating less frequent administration. In addition, targeting of IL-23p19 may alter the circulating level of IL-12p40, which is a subunit of both IL-23 and IL-12, and also appears to exist in a p40 homodimer form. Because the potential biological effects of alterations in the levels of IL-12 and/or IL-12p40 homodimer are not fully understood, it would be preferable to block IL-23 signaling in a way that doesn't involve targeting the soluble cytokine (IL-23), but instead targets the IL-23-specific receptor subunit IL-23R.

### III. Generation of IL-23R Specific Antibodies

Any suitable method for generating monoclonal antibodies may be used. For example, recipient may be immunized with IL-23R. or a fragment thereof. Any suitable method of immunization can be used. Such methods can include adjuvants, other immunostimulants, repeated booster immunizations, and the use of one or more immunization routes. Any suitable source of IL-23R can be used as the immunogen for the generation of the non-human antibody of the compositions and methods disclosed herein. Such forms include, but are not limited whole protein, peptide(s), and epitopes generated through recombinant, synthetic, chemical or enzymatic degradation means known in the art. In preferred embodiments the immunogen comprises the extracellular portion of IL-23R. The domain structure of IL-23R is discussed in U.S. Pat. No. 6,756,481, in which the extracellular domain is predicted to comprise amino acids 1-328 of the mature form of human IL-23R, corresponding to residues 24-351 of SEQ ID NO: 9 of the present application. See also WO 2004/052157. The extracellular domain may be defined differently depending on the proposed purpose, such as design of an immunogenic polypeptide construct.

Any form of the antigen can be used to generate the antibody that is sufficient to generate a biologically active antibody. Thus, the eliciting antigen may be a single epitope, multiple epitopes, or the entire protein alone or in combination with one or more immunogenicity enhancing agents known in the art. The eliciting antigen may be an isolated full-length protein, a cell surface protein (e.g., immunizing with cells transfected with at least a portion of the antigen), or a soluble protein (e.g., immunizing with only the extracellular domain portion of the protein). The antigen may be produced in a genetically modified cell. The DNA encoding the antigen may genomic or non-genomic (e.g., cDNA) and encodes at least a portion of the extracellular domain. As used herein, the term “portion” refers to the minimal number of amino acids or nucleic acids, as appropriate, to constitute an immunogenic epitope of the antigen of interest. Any genetic vectors suitable for transformation of the cells of interest may be employed, including but not limited to adenoviral vectors, plasmids, and non-viral vectors, such as cationic lipids.

Any suitable method can be used to elicit an antibody with the desired biologic properties to inhibit IL-23/IL-23R. It is desirable to prepare monoclonal antibodies (mAbs) from various mammalian hosts, such as mice, rats, other rodents, humans, other primates, etc. Description of techniques for preparing such monoclonal antibodies may be found in, e.g., Stites et al. (eds.) BASIC AND CLINICAL IMMUNOLOGY (4th ed.) Lange Medical Publications. Los Altos, Calif., and references cited therein; Harlow and Lane (1988) ANTIBODIES: A LABORATORY MANUAL CSH Press; Goding (1986) MONOCLONAL ANTIBODIES: PRINCIPLES AND PRACTICE (2d ed.) Academic Press, New York, N.Y. Thus, monoclonal antibodies may be obtained by a variety of techniques familiar to researchers skilled in the art. Typically, spleen cells from an animal immunized with a desired antigen are immortalized, commonly by fusion with a myeloma cell. See Kohler and Milstein (1976) Eur. J. Immunol. 6:511-519. Alternative methods of immortalization include transformation with Epstein Barr Virus, oncogenes, or retroviruses, or other methods known in the art. See, e.g., Doyle et al. (eds. 1994 and periodic supplements) CELL AND TISSUE CULTURE: LABORATORY PROCEDURES, John Wiley and Sons, New York, N.Y. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate DNA sequences that encode a monoclonal antibody or a antigen binding fragment thereof by screening a DNA library from human B cells according, e.g., to the general protocol outlined by Huse et al. (1989) Science 246:1275-1281.

Other suitable techniques involve selection of libraries of antibodies in phage or similar vectors. See, e.g., Huse et al. supra; and Ward et al. (1989) Nature 341:544-546. The polypeptides and antibodies of the present invention may be used with or without modification, including chimeric or humanized antibodies. Frequently, the polypeptides and antibodies will be labeled by joining, either covalently or non-covalently, a substance that provides for a detectable signal. A wide variety of labels and conjugation techniques are known and are reported extensively in both the scientific and patent literature. Suitable labels include radionuclides, enzymes, substrates, cofactors, inhibitors, fluorescent moieties, chemiluminescent moieties, magnetic particles, and the like. Patents teaching the use of such labels include U.S. Pat. Nos. 3,817,837; 3,850,752; 3,939,350; 3,996,345; 4,277,437; 4,275,149; and 4.366,241. Also, recombinant immunoglobulins may be produced, see Cabilly U.S. Pat. No. 4,816,567; and Queen et al. (1989) Proc. Nat'l Acad. Sci. (ISA 86:10029-10033; or made in transgenic mice, see Mendez et al. (1997) Nature Genetics 15:146-156. See also Abgenix and Medarex technologies.

Antibodies or binding compositions against predetermined fragments of IL-23R can be raised by immunization of animals with conjugates of the polypeptide, fragments, peptides, or epitopes with carrier proteins. Monoclonal antibodies are prepared from cells secreting the desired antibody. These antibodies can be screened for binding to normal or defective IL-23R. These monoclonal antibodies will usually bind with at least a K_{d} of about 1 μM, more usually at least about 300 nM, 30 nM, 10 nM, 3 nM, 1 nM, 300 pM, 100 pM, 30 pM, 10 pM, 1 pM or better, usually determined by ELISA or BIAcore® label-free interaction analysis system. Suitable non-human antibodies may also be identified using the biologic assays described in Examples 5, 6 and 8, below.

A hybridoma expressing anti-human IL-23R antibody 8B10 (rat IgG2a kappa) was deposited pursuant to the Budapest Treaty with American Type Culture Collection (ATCC—Manassas, Va., USA) on Aug. 17, 2006 under Accession Number PTA-7800.

### IV. Humanization of IL-23R Specific Antibodies

Any suitable non-human antibody can be used as a source for the hypervariable region. Sources for non-human antibodies include, but are not limited to, murine (e.g. Mus musculus), rat (e.g. Rattus norvegicus), Lagomorphs (including rabbits), bovine, and primates. For the most part, humanized antibodies are human immunoglobulins (recipient antibody) in which hypervariable region residues of the recipient are replaced by hypervariable region residues from a non-human species (donor antibody) such as mouse, rat, rabbit or non-human primate having the desired specificity, affinity, and capacity. In some instances, Fv framework region (FR) residues of the human immunoglobulin are replaced by corresponding non-human residues. Furthermore, humanized antibodies may comprise residues that are not found in the recipient antibody or in the donor antibody. These modifications are made to further refine antibody performance of the desired biological activity. For further details, see Jones et al. (1986) Nature 321:522-525; Reichmann et al. (1988) Nature 332:323-329; and Presta (1992) Curr. Op. Struct. Biol. 2:593-596.

Methods for recombinantly engineering antibodies have been described, e.g., by Boss et al. (U.S. Pat. No. 4,816,397), Cabilly et al. (U.S. Pat. No. 4,816,567), Law et al. (European Patent Application Publication No. EP438310A1) and Winter (European Patent Application Publication No. EP239400B1).

Amino acid sequence variants of humanized anti-IL-23R antibody are prepared by introducing appropriate nucleotide changes into the humanized anti-IL-23R antibody DNA, or by peptide synthesis. Such variants include, for example, deletions from, and/or insertions into, and/or substitutions of, residues within the amino acid sequences shown for the humanized anti-IL-23R. antibody. Any combination of deletion, insertion, and substitution is made to arrive at the final construct, provided that the final construct possesses the desired characteristics. The amino acid changes also may alter post-translational processes of the humanized anti-IL-23R antibody, such as changing the number or position of glycosylation sites.

A useful method for identification of certain residues or regions of the humanized anti-IL-23R antibody polypeptide that are preferred locations for mutagenesis is called “alanine scanning mutagenesis,” as described by Cunningham and Wells (1989) Science 244: 1081-1085. Here, a residue or group of target residues are identified (e.g., charged residues such as Arg, Asp, His, Lys, and Glu) and replaced by a neutral or negatively charged amino acid (most preferably alanine or polyal.anine) to affect the interaction of the amino acids with IL-23R antigen. The amino acid residues demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, Ala scanning or random mutagenesis is conducted at the target codon or region and the expressed humanized anti-IL-23R antibody variants are screened for the desired activity.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include humanized anti-IL-23R antibody with an N-terminal methionyl residue or the antibody fused to an epitope tag. Other insertional variants of the humanized anti-IL-23R antibody molecule include the fusion to the N- or C-terminus of humanized anti-IL-23R antibody of an enzyme or a polypeptide that increases the serum half-life of the antibody.

Another type of variant is an amino acid substitution variant. These variants have at least one amino acid residue in the humanized anti-IL-23R antibody molecule removed and a different residue inserted in its place. The sites of greatest interest for substitutional mutagenesis include the hypervariable loops, but FR alterations are also contemplated.

Another type of amino acid variant of the antibody alters the original glycosylation pattern of the antibody. By altering is meant deleting one or more carbohydrate moieties found in the antibody, and/or adding one or more glycosylation sites that are not present in the antibody. Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the above-described tripeptide sequences (for N-linked glycosylation sites). The alteration may also be made by the addition of or substitution by, one or more serine or threonine residues to the sequence of the original antibody (for O-linked glycosylation sites).

Yet another type of amino acid variant is the substitution of residues to provide for greater chemical stability of the final humanized antibody. For example, an asparagine (N) residue may be changed to reduce the potential for formation of isoaspartate at any NG sequences within a rodent CDR. A similar problem may occur at a DO sequence. Reissner and Aswad (2003) Cell. Mol. Life Sci. 60:1281. Isoaspartate formation may debilitate or completely abrogate binding of an antibody to its target antigen. Presta (2005) J. Allergy Clin. Immunol. 116:731 at 734. In one embodiment, the asparagine is changed to glutamine (Q). It may also be desirable to alter an amino acid adjacent to an asparagine (N) or glutamine (Q) residue to reduce the likelihood of deamidation, which occurs at greater rates when small amino acids occur adjacent to asparagine or glutamine. Bischoff & Kolbe (1994) J. Chromatog. 662:261. In addition, methionine residues in rodent CDRs may be changed to reduce the possibility that the methionine sulfur would oxidize, which could reduce antigen binding affinity and also contribute to molecular heterogeneity in the final antibody preparation. Id. In one embodiment, the methionine is changed to alanine (A). Antibodies with such substitutions are subsequently screened to ensure that the substitutions do not decrease IL-23R binding affinity to unacceptable levels.

Specifically, with regard to antibody hu8B10 and hu8B10-b, several sequence variants may improve stability. Accordingly, with regard to deamidation, the invention encompasses VH comprising any combination or permutation of N30S, N30Q, N30D, N52S, N52Q, N52D and N57S, N57Q, N57D, and/or VL comprising any combination or permutation of N32S, N32Q or N32D. With regard to isoaspartate formation, the invention encompasses VL comprising D56S or D56E. For example, antibody hu8B10-c incorporates CDRH1 variant N30S and CDRL2 variant D56S.

Nucleic acid molecules encoding amino acid sequence variants of humanized IL-23R specific antibody are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of humanized anti-IL-23R antibody.

Ordinarily, amino acid sequence variants of the humanized anti-IL-23R antibody will have an amino acid sequence having at least 75% amino acid sequence identity with the original humanized antibody amino acid sequences of either the heavy or the light chain more preferably at least 80%, more preferably at least 85%, more preferably at least 90%, and most preferably at least 95%, 98%, or 99%. identity or homology with respect to this sequence is defined herein as the percentage of amino acid residues in the candidate sequence that are identical with the humanized anti-IL-23R residues, after aligning the sequences and introducing gaps, if necessary, to achieve the maximum percent sequence identity, and not considering any conservative substitutions as part of the sequence identity. None of N-terminal, C-terminal, or internal extensions, deletions, or insertions into the antibody sequence shall be construed as affecting sequence identity or homology.

Structure-function data are provided herein for anti-IL-23R antibody hu8B10-b as follows. One of skill in the art would recognize that alteration of the CDR sequences would be expected to have the most dramatic effects on antigen-binding affinity. Murphy et al., Janeway's Immunobiology, Seventh Ed., 2008, Chapter 3. The CDR regions for hu8B10-b are indicated at FIGS. 1 and 2. In addition, site-directed mutagenesis was performed at various positions in the heavy and light chain variable domains to discover high affinity variants of antibody hu8B10, as described in greater detail at Example 7 and Table 2. These sequence variants include heavy chain variable domain S31A, Y32.A, G33A, A49G, D50A, 151A, N52A, S53A, K54A, S55A, Y56A, Y58G, Y61A, R74A, H101A, H102A, H102W, S103A, D104A, Y105A, F106A, E107A, and light chain variable domain E27A, D28A, Y30A, N31A, N31del, N32A, Y49A, H50A, S53A, D91A, S92A, E93A, Y94A. In addition, various mutations of antibody hu8B10-b were made within the variable domains to maximize binding affinity for the target (human IL-23R) and to increase stability, as described in greater detail at Example 8. These sequence variants include heavy chain variable domain E46D, W47L, R100V, N30S, N30Q, N30D, N52S, N52Q, N52D, N57S, N57Q, N57D, and light chain variable domain L24R, E27Q, F71Y, N32S, N32Q, N32D, D56S, D56E. Affinity data for several of these variants are provided at Table 3.

The humanized antibody can be selected from any class of immunoglobulins, including IgM, IgG, IgD, IgA, and IgE. Preferably, the antibody is an IgG antibody. Any isotype of IgG can be used, including IgG₁, IgG₂, IgG₃, and IgG₄. Variants of the IgG isotypes are also contemplated. The humanized antibody may comprise sequences from more than one class or isotype. Optimization of the necessary constant domain sequences to generate the desired biologic activity is readily achieved by screening the antibodies in the biological assays described in the Examples.

Likewise, either class of light chain can be used in the compositions and methods herein. Specifically, kappa, lambda, or variants thereof are useful in the present compositions and methods.

Any suitable portion of the CDR sequences from the non-human antibody can be used. The CDR sequences can be mutagenized by substitution, insertion or deletion of at least one residue such that the CDR sequence is distinct from the human and non-human antibody sequence employed. It is contemplated that such mutations would be minimal. Typically, at least 75% of the humanized antibody residues will correspond to those of the non-human CDR residues, more often 90%, and most preferably greater than 95%.

Any suitable portion of the FR sequences from the human antibody can be used. The FR sequences can be mutagenized by substitution, insertion or deletion of at least one residue such that the FR sequence is distinct from the human and non-human antibody sequence employed. It is contemplated that such mutations would be minimal. Typically, at least 75% of the humanized antibody residues will correspond to those of the human FR residues, more often 90%, and most preferably greater than 95%, 98% or 99%.

CDR and FR residues are determined according to the standard sequence definition of Kabat. Kabat et al. (1987) Sequences of Proteins of immunological Interest, National institutes of Health, Bethesda Md. FIGS. 1 and 2 provide sequence lineups of heavy and light chain variable domains of the antibodies of the present invention, indicating the CDRs. SEQ ID NO: 1 is the heavy chain variable domain sequence of rat anti-human IL-23R antibody 8B10, and SEQ ID NO: 2 is the corresponding light chain variable domain sequence. SEQ ID NOs: 11, 13 and 22 are heavy chain variable domain sequences of three humanized forms of 8B10, and SEQ ID NOs: 12, 14 and 23 are the corresponding light chain variable domain sequences. For the heavy chains, CDRH1 is residues 26-35, CDRH2 is residues 50-68 and CDRH3 is residues 101-108 of the corresponding heavy chain variable domain sequence. For the light chain variable domains, CDRL1 is residues 24-34, CDRL2 is residues 50-56 and CDRH3 is residues 89-97 of the corresponding light chain variable domain sequence. The individual CDR sequences are presented at SEQ ID NOs: 3-8, 15, 20 and 21, as indicated in Table 4. CDRs are identical for rat 8B10 and hu8B10, and differ in hu8B10-b and hu8B10-c as indicated in the FIGS. 1 and 2.

In one embodiment, CDRs include variants of any single sequence CDR disclosed herein (SEQ ID NOs: 3-8, 15, 20 and 21), in which the variant comprises 1, 2, 3, 4, 5 or more conservative amino acid substitutions relative to the disclosed sequence, as determined using Table 1.

Heavy and light chain sequences (SEQ ID NOs: 16, 17, 24, 25 and 26) are provided without signal sequences. Exemplary heavy and light chain signal sequences are provided at SEQ ID NOs: 18 and 19, respectively. The signal sequences, or nucleic acid sequences encoding the signal sequences, may be appended to the N-terminus of the respective antibody chains to create a precursor protein for secretion from a host cell. Alternative signal sequences may also be used, and several can be found at SPdb:a Signal Peptide Database. Choo et al. (2005) BMC Bioinformatics 6:249.

Also contemplated are chimeric antibodies. As noted above, typical chimeric antibodies comprise a portion of the heavy and/or light chain identical with, or homologous to, corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies. so long as they exhibit the desired biological activity. See U.S. Pat. No. 4,816,567; and Morrison et al. (1984) Proc. Natl. Acad. Sci. USA 81: 6851-6855.

One bispecific agent encompassed by the present invention is the bispecific T cell engager (BiTE). Bauerle et al. (2008) Drugs of the Future 33:137; Mack et al. (1995) Proc. Nat'l Acad. Sci. (USA) 92:7021. Such antibodies bind to a marker on a target cell (e.g. IL-23R) and a marker on cytotoxic T lymphocytes (e.g. CD3) to tether the cytotoxic T cells in proximity to the desired target cell, thus enhancing the apoptosis of the target cells. One such antibody, blinatumomab, has shown promise in early clinical trials for treatment of non-Hodgkin's lymphoma, and appears to be efficacious at very low doses. Bargou et al. (2008) Science 321:974.

In yet other embodiments, different constant domains may be appended to humanized V_{L} and V_{H} regions derived from the CDRs provided herein. For example, if a particular intended use of an antibody (or fragment) of the present invention were to call for altered effector functions, a heavy chain constant domain other than IgG1 may be used. An exemplary human IgG1 constant domain sequence is provided at residues 120-449 of SEQ ID NO: 16.

Although IgG1 antibodies provide for long half-life and for effector functions, such as complement activation and antibody-dependent cellular cytotoxicity, such activities may not be desirable for all uses of the antibody. In such instances an IgG4 constant domain, for example, may be used, An exemplary human IgG4 constant domain sequence is provided at residues 120-446 of SEQ ID NO: 26. This IgG4 constant domain differs from the native human IgG4 constant domain (Swiss-Prot Accession No. P01861.1, the disclosure of which is hereby incorporated by reference) at position 108, where the native Ser108 is replaced with Pro to prevent a potential inter-chain disulfide bond between Cys106 and Cys109 that could interfere with proper intra-chain disulfide bond formation. See Angal et al. (1993) Mol. Immunol. 30:105.

In various embodiments, the anti-IL-23R antibodies comprise heavy and light chain combinations such as SEQ ID NOs: 16 and 17 (hu8B10-b, IgG1), SEQ ID NOs: 24 and 25 (hu8B10-c, IgG1), and SEQ ID NOs: 25 and 26 (hu8B10-c., IgG4). Other pairwise combinations of heavy chains (SEQ ID NOs: 16, 24 and 26) and light chains (SEQ ID NOs: 17 and 25) are possible.

Exemplary nucleic acid sequences encoding the heavy and light chains of the hu8B10-c IgG4 embodiment of the present invention., including residues encoding the signal sequences of SEQ ID NOs: 18 and 19, are provided at SEQ ID NOs: 27 and 28, respectively. The signal sequences may be replaced with different signal sequences without affecting the mature sequence of the antibody chains, and thus the sequences encoding the signal sequences are not essential. Accordingly, in another embodiment, the invention relates to these same nucleic acid sequences without the signal sequences, i.e. residues 58-1395 of SEQ ID NO: 27 and residues 58-699 of SEQ ID NO: 28.

### V. Biological Activity of Humanized Anti-IL-23R Antibodies

Antibodies having the characteristics identified herein as being desirable in a humanized anti-IL-23R antibody can be screened for inhibitory biologic activity in vitro or suitable binding affinity. Antagonist antibodies may be distinguished from agonist antibodies using the biological assays provided at Examples 5, 6 and 7. Antibodies that exhibit agonist activity will not block the activity of IL-23, but will instead stimulate the response typically caused by IL-23, such as increasing cell proliferation in the Ba/F3 assay of Example 5 and/or increasing IL-17 production in the splenocyte assay of Example 6. Although agonist antibodies may find use in some therapeutic indications, the anti-IL-23R. antibodies disclosed herein are intended to be antagonist antibodies unless otherwise indicated.

To screen for antibodies that bind to the epitope on human IL-23R bound by an antibody of interest (e.g., those that block binding of IL-23), a routine cross-blocking assay such as that described in ANTIBODIES, A LABORATORY MANUAL, Cold Spring Harbor Laboratory, Ed Harlow and David Lane (1988), can be performed. Antibodies that bind to the same epitope are likely to cross-block in such assays, but not all cross-blocking antibodies will necessarily bind at precisely the same epitope since cross-blocking may result from steric hindrance of antibody binding by antibodies bind at overlapping epitopes, or even nearby non-overlapping epitopes.

Alternatively, epitope mapping, e.g., as described in Champe et at. (1995) J. Biol. Chem. 270:1388-1394, can be performed to determine whether the antibody binds an epitope of interest. “Alanine scanning mutagenesis,” as described by Cunningham and Wells (1989) Science 244: 1081-1085, or some other form of point mutagenesis of amino acid residues in human IL-23R may also be used to determine the functional epitope for an anti-IL-23R antibody of the present invention. Mutagenesis studies, however, may also reveal amino acid residues that are crucial to the overall three-dimensional structure of IL-23R but that are not directly involved in antibody-antigen contacts, and thus other methods may be necessary to confirm a. functional epitope determined. using this method.

The epitope bound by a specific antibody may also be determined by assessing binding of the antibody to peptides comprising fragments of human IL-23R (SEQ ID NO: 9). A series of overlapping peptides encompassing the sequence of IL-23R may be synthesized and screened for binding, e.g. in a direct ELISA, a competitive ELISA (where the peptide is assessed for its ability to prevent binding of an antibody to IL-23R bound to a well of a microtiter plate), or on a chip. Such peptide screening methods may not be capable of detecting some discontinuous functional epitopes, i.e. functional epitopes that involve amino acid residues that are not contiguous along the primary sequence of the IL-23R polypeptide chain.

The epitope bound by antibodies of the present invention may also be determined by structural methods, such as X-ray crystal structure determination (e.g., WO 2005/044853), molecular modeling and nuclear magnetic resonance (NMR) spectroscopy, including NMR determination of the H-D exchange rates of labile amide hydrogens in IL-23R when free and when bound in a complex with an antibody of interest (Zinn-Justin et al. (1992) Biochemistry 31:11335-11347; Zinn-Justin et al. (1993) Biochemistry 32:6884-6891).

With regard to X-ray crystallography, crystallization may be accomplished using any of the known methods in the art (e.g. Giege et al. (1994) Acta Crystallogr. D50:339-350; McPherson (1990) Eur. J. Biochem. 189:1-23), including microbatch (e.g. Chayen (1997) Structure 5:1269-1274), hanging-drop vapor diffusion (e.g. McPherson (1976) J. Biol. Chem. 251:6300-6303), seeding and dialysis. It is desirable to use a protein preparation having a concentration of at least about 1 mg/mL. and preferably about 10 mg/mL to about 20 mg/mL. Crystallization may be best achieved in a precipitant solution containing polyethylene glycol 1000-20,000 (PEG; average molecular weight ranging from about 1000 to about 20,000 Da), preferably about 5000 to about 7000 Da, more preferably about 6000 Da, with concentrations ranging from about 10% to about 30% (w/v). It may also be desirable to include a protein stabilizing agent, e.g. glycerol at a concentration ranging from about 0.5% to about 20%. A suitable salt, such as sodium chloride, lithium chloride or sodium citrate may also be desirable in the precipitant solution, preferably in a concentration ranging from about 1 mM to about 1000 mM. The precipitant is preferably buffered to a pH of from about 4,0 to about 10.0, preferably about 7.0 to 8.5, e.g. 8.0. Specific buffers useful in the precipitant solution may vary and are well-known in the art. Scopes, Protein Purification: Principles and Practice. Third ed., (1994) Springer-Verlag, New York. Examples of useful buffers include, but are not limited to, HEPES, Tris, MES and acetate. Crystals may be grow at a wide range of temperatures, including 2° C., 4° C., 8° C. and 26° C.

Antibody:antigen crystals may be studied using well-known X-ray diffraction techniques and may be refined using computer software such as X-PLOR (Yale University, 1992, distributed by Molecular Simulations, Inc.; see e.g. Blundell & Johnson (1985) Meth. Enzymol. 114 & 115, H. W. Wyckoff et al. eds., Academic Press; U.S. Patent Application Publication No. 2004/0014194), and BUSTER (Bricogne (1993) Acta Cryst. D49:37-60; Bricogne (1997) Meth. Enzymol. 276A:361-423, Carter & Sweet, eds.; Roversi et al. (2000) Acta Cryst. D56:1313-1323).

Additional antibodies binding to the same epitope as an antibody of the present invention may be obtained, for example, by screening of antibodies raised against IL-23R for binding to the epitope, or by immunization of an animal with a peptide comprising a fragment of human IL-23R comprising the epitope sequence. Antibodies that bind to the same functional epitope might be expected to exhibit similar biological activities, such as blocking receptor binding, and such activities can be confirmed by functional assays of the antibodies.

Antibody affinities may be determined using standard analysis. See, e.g., Examples 3 and 4. Preferred humanized antibodies are those that bind human IL-23R with a K_{d} value of no more than about 1×10⁻⁷ M; preferably no more than about 1×10⁻⁸ M; more preferably no more than about 1×10⁻⁹ M; and most preferably no more than about 1×10⁻¹⁰ M or even 1×10⁻¹¹ M.

The antibodies and fragments thereof useful in the present compositions and methods are biologically active antibodies and fragments. As used herein, the term “biologically active” refers to an antibody or antibody fragment that is capable of binding the desired the antigenic epitope and directly or indirectly exerting a biologic effect. Typically, these effects result from the failure of IL-23 to bind IL-23 receptor. As used herein, the term “specific” refers to the selective binding of the antibody to the target antigen epitope. Antibodies can be tested for specificity of binding by comparing binding to IL-23R to binding to irrelevant antigen or antigen mixture under a given set of conditions. if the antibody binds to IL-23R at least 10, and preferably 50 times more than to irrelevant antigen or antigen mixture then it is considered to be specific. An antibody that “specifically binds” to IL-23R does not bind to proteins that do not comprise the IL-23R-derived sequences, i.e. “specificity” as used herein relates to IL-23R specificity, and not any other sequences that may be present in the protein in question. For example, as used herein, an antibody that “specifically binds” to a polypeptide comprising IL-23R will typically bind to FLAG®-hIL-23R, which is a fusion protein comprising IL-23R and a FLAG® peptide tag, but it does not bind to the FLAG® peptide tag alone or when it is fused to a protein other than IL-23R.

IL-23R-specific binding compounds of the present invention, such as inhibitory IL-23R specific antibodies, can inhibit its biological activity in any manner, including but not limited to reducing production of IL-1β and TNF by peritoneal macrophages and IL-17 by Th17 T cells. See Langrish el al. (2004) Immunol. Rev. 202:96-105. Anti-IL-23R antibodies will also be able to inhibit the gene expression of IL-17A, IL-17F, CCL7, CCL17, CCL20, CCL22, CCR1, and GM-CSF. See Langrish et al. (2005) J. Exp. Med. 201:233-240. IL-23R-specific binding compounds of the present invention, such as anti IL-23R antibodies, will also block the ability of IL-23 to enhance proliferation or survival of Th17 cells. Cua and Kastelein (2006) Nat. Immunol. 7:557-559. The inhibitory activity of anti-IL-23R antibodies will be useful in the treatment of inflammatory, autoimmune, and proliferative disorders. Examples of such disorders are described in PCT patent application publications WO 04/081190; WO 04/071517; WO 00/53631; and WO 01/18051.

### VI. Pharmaceutical Compositions

To prepare pharmaceutical or sterile compositions including IL-23R antibody, the cytokine analogue or mutein, antibody thereto, or nucleic acid thereof is admixed with a pharmaceutically acceptable carrier or excipient. See, e.g., Remington's Pharmaceutical Sciences and U.S. Pharmacopeia: National Formulary, Mack Publishing Company, Easton, Pa. (1984).

Formulations of therapeutic and diagnostic agents may be prepared by mixing with physiologically acceptable carriers, excipients, or stabilizers in the form of, e.g., lyophilized powders, slurries, aqueous solutions or suspensions. See, e.g., Hardman et al. (2001) Goodman and Gilman's The Pharmacological Basis of Therapeutics, McGraw-Hill, New York, N.Y.; Gennaro (2000) Remington: The Science and Practice of Pharmacy, Lippincott, Williams, and Wilkins, New York, N.Y.; Avis et al, (eds.) (1993) Pharmaceutical Dosage Forms: Parenteral Medications, Marcel Dekker, NY.; Lieberman, et al. (eds.) (1990) Pharmaceutical Dosage Forms: Tablets, Marcel Dekker, NY.; Lieberman et al. (eds.) (1990) Pharmaceutical Dosage Forms: Disperse Systems, Marcel Dekker, NY.; Weiner and Kotkoskie (2000) Excipient Toxicity and Safety, Marcel Dekker, Inc., New York, N.Y.

Toxicity and therapeutic efficacy of the antibody compositions, administered alone or in combination with an immunosuppressive agent, can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio of LD₅₀ to ED₅₀. Antibodies exhibiting high therapeutic indices are preferred. The data obtained from these cell culture assays and animal studies can be used in formulating a range of dosage for use in human. The dosage of such compounds lies preferably within a range of circulating concentrations that include the ED₅₀ with little or no toxicity. The dosage may vary within this range depending upon the dosage form employed and the route of administration.

Suitable routes of administration may, for example, include oral, rectal, transmucosal, or intestinal administration; parenteral delivery, including intramuscular, intradermal, subcutaneous, intramedullary injections, as well as intrathecal, direct intraventricular, intravenous, intraperitoneal, intranasal, or intraocular injections. Administration of antibody used in the pharmaceutical composition or to practice the method of the present invention can be carried out in a variety of conventional ways, such as oral ingestion, inhalation, topical application or cutaneous, subcutaneous, intraperitoneal, parenteral, intraarterial or intravenous injection.

Alternately, one may administer the antibody in a local rather than systemic manner, for example, via injection of the antibody directly into an arthritic joint or pathogen-induced lesion characterized by immunopathology, often in a depot or sustained release formulation. Furthermore, one may administer the antibody in a targeted drug delivery system, for example, in a liposome coated with a tissue-specific antibody, targeting, for example, arthritic joint or pathogen-induced lesion characterized by immunopathology. The liposomes will be targeted to and taken up selectively by the afflicted tissue.

Selecting an administration regimen for a therapeutic depends on several factors, including the serum or tissue turnover rate of the entity, the level of symptoms, the immunogenicity of the entity, and the accessibility of the target cells in the biological matrix. Preferably, an administration regimen maximizes the amount of therapeutic delivered to the patient consistent with an acceptable level of side effects. Accordingly, the amount of biologic delivered depends in part on the particular entity and the severity of the condition being treated. Guidance in selecting appropriate doses of antibodies, cytokines, and small molecules are available. See, e.g., Wawrzynczak (1996) Antibody Therapy, Bios Scientific Pub. Ltd, Oxfordshire, UK; Kresina (ed.) (1991) Monoclonal Antibodies, Cytokines and Arthritis, Marcel Dekker, New York, N.Y.; Bach (ed.) (1993) Monoclonal Antibodies and Peptide Therapy in Autoimmune Diseases, Marcel Dekker, New York, N.Y.; Baert et al. (2003) New. Engl. J. Med 348:601-608; Milgrom et al. (1999) New Engl. J. Med 341:1966-1973; Slamom et al. (2001) New Engl. J. Med. 344:783-792; Beniaminovitz et al. (2000) New Engl. J. Med. 342:613-619; Ghosh et al. (2003) New Engl. J. Med. 348:24-32; Lipsky et al. (2000) New Engl. J. Med 343:1594-1602.

Determination of the appropriate dose is made by the clinician, e.g., using parameters or factors known or suspected in the art to affect treatment or predicted to affect treatment. Generally, the dose begins with an amount somewhat less than the optimum dose and it is increased by small increments thereafter until the desired or optimum effect is achieved relative to any negative side effects. Important diagnostic measures include those of symptoms of, e.g., the inflammation or level of inflammatory cytokines produced. Preferably, a biologic that will be used is substantially derived from the same species as the animal targeted for treatment (e.g. a humanized antibody for treatment of human subjects), thereby minimizing any immune response to the reagent.

Antibodies, antibody fragments, and cytokines can be provided by continuous infusion, or by doses at intervals of e.g., one day, 1-7 times per week, one week, two weeks, monthly, bimonthly, etc. Doses may be provided, e.g., intravenously, subcutaneously, topically, orally, nasally, rectally, intramuscular, intracerebrally, intraspinally, or by inhalation. A preferred dose protocol is one involving the maximal dose or dose frequency that avoids significant cant undesirable side effects. A total weekly dose is generally at least 0.05 μg/kg, 0.2 μg/kg, 0.5 μg/kg, 1 μg/kg, 10 μg/kg, 100 μg/kg, 0.2 mg/kg, 1.0 mg/kg, 2.0 mg/kg, 10 mg/kg, 25 mg/kg, 50 mg/kg body weight or more. See, e.g., Yang et al. (2003) New Engl. J. Med. 349:427-434; Herold et al. (2002) New Engl. J. Med. 346:1692-1698; Liu et al. (1999) J. Neurol. Neurosurg. Psych, 67:451-456; Portielji et al. (20003) Cancer Immunol. Immunother. 52:133-144. The desired dose of a small molecule therapeutic, e.g., a peptide mimetic, natural product, or organic chemical, is about the same as for an antibody or polypeptide, on a moles/kg basis. Even lower doses may be used for particularly efficacious embodiments of the present invention, such as BiTE antibodies.

Methods for co-administration or treatment with a second therapeutic agent, e.g., a cytokine, antibody, steroid, chemotherapeutic agent, antibiotic, or radiation, are well known in the art, see, e.g., Hardman et al. (eds.) (2001) Goodman and Gilman's The Pharmacological Basis of Therapeutics, 10^{th} ed., McGraw-Hill, New York, N.Y.; Poole and Peterson (eds.) (2001) Pharmacotherapeutics for Advanced Practice: A Practical Approach, Lippincott, Williams & Wilkins, Phila., Pa.; Chabner and Longo (eds.) (2001) Cancer Chemotherapy and Biotherapy, Lippincott, Williams & Wilkins, Phila., Pa. The pharmaceutical composition of the invention may also contain other immunosuppressive or immunomodulating agents. Any suitable immunosuppressive agent can be employed, including but not limited to anti-inflammatory agents, corticosteroids, cyclosporine, tacrolimus (i.e., FK-506), sirolimus, interferons, soluble cytokine receptors (e.g., sTNRF and sIL-1R), agents that neutralize cytokine activity (e.g., inflixmab, etanercept), mycophenolate mofetil, 15-deoxyspergualin, thalidomide, glatiramer, azathioprine, leflunomide, cyclophosphamide, methotrexate, and the. like. The pharmaceutical composition can also be employed with other therapeutic modalities such as phototherapy and radiation.

Typical veterinary, experimental, or research subjects include monkeys, dogs, cats, rats, mice, rabbits, guinea pigs, horses, and humans.

### VII. Antibody Production

In one embodiment, for recombinant production of the antibodies of the present invention, the nucleic acids encoding the two chains are isolated and inserted into one or more replicable vectors for further cloning (amplification of the DNA) or for expression. DNA encoding the monoclonal antibody is readily isolated and sequenced using conventional procedures (e.g., by using oligonucleotide probes that are capable of binding specifically to genes encoding the heavy and light chains of the antibody). Many vectors are available. The vector components generally include, but are not limited to, one or more of the following: a signal sequence, an origin of replication, one or more marker genes, an enhancer element, a promoter, and a transcription termination sequence. In one embodiment, both the light and heavy chains of a humanized anti-IL-23.R. antibody of the present invention are expressed from the same vector, e.g. a plasmid or an adenoviral vector.

Antibodies of the present invention may be produced by any method known in the art. I.n one embodiment, antibodies are expressed in mammalian or insect cells in culture, such as chinese hamster ovary (CHO) cells, human embryonic kidney (HEK) 293 cells, mouse myeloma NSO cells, baby hamster kidney (BHK) cells, Spodoptera frugiperda ovarian (Sf9) cells. In one embodiment, antibodies secreted from CHO cells are recovered and purified by standard chromatographic methods, such as protein A, cation exchange, anion exchange, hydrophobic interaction, and hydroxyapatite chromatography. In one embodiment, resulting antibodies are concentrated and stored in 20 mM sodium acetate, pH 5.5.

In another embodiment, the antibodies of the present invention are produced in yeast according to the methods described in WO 2005/040395. Briefly, vectors encoding the individual light or heavy chains of an antibody of interest are introduced into different yeast haploid cells, e.g. different mating types of the yeast Pichia pastoris, which yeast haploid cells are optionally complementary auxotrophs. The transformed haploid yeast cells can then be mated or fused to give a diploid yeast cell capable of producing both the heavy and the light chains. The diploid strain is then able to secret the fully assembled and biologically active antibody. The relative expression levels of the two chains can be optimized, for example, by using vectors with different copy number, using transcriptional promoters of different strengths, or inducing expression from inducible promoters driving transcription of the genes encoding one or both chains.

In one embodiment, the respective heavy and light chains of a plurality of different anti-IL-23R antibodies (the “original” antibodies) are introduced into yeast haploid cells to create a library of haploid yeast strains of one mating type expressing a plurality of light chains, and a library of haploid yeast strains of a different mating type expressing a plurality of heavy chains. These libraries of haploid strains can be mated (or fused as spheroplasts) to produce a series of diploid yeast cells expressing a combinatorial library of antibodies comprised of the various possible permutations of light and heavy chains. The combinatorial library of antibodies can then be screened to determine whether any of the antibodies has properties that are superior (e.g. higher affinity for IL-23R) to those of the original antibodies. See. e.g., WO 2005/040395.

In another embodiment, antibodies of the present invention are human domain antibodies in which portions of an antibody variable domain are linked in a polypeptide of molecular weight approximately 13 kDa. See, e.g., U.S. Pat. Publication No. 2004/0110941. Such single domain, low molecular weight agents provide numerous advantages in terms of ease of synthesis, stability, and route of administration.

### VIII. Uses

The present invention provides methods for using anti-IL-23R antibodies and fragments thereof for the treatment and diagnosis of inflammatory disorders and conditions, e.g., of the central nervous system, peripheral nervous system, and gastrointestinal tract, as well as autoimmune and proliferative disorders.

Methods are provided for the treatment of, e.g., multiple sclerosis (MS), including relapsing-remitting MS and primary progressive MS, Alzheimer's disease, amyotrophic lateral sclerosis (a.k.a. ALS; Lou Gehrig's disease), ischemic brain injury, prion diseases, and HIV-associated dementia. Also provided are methods for treating neuropathic pain, posttraumatic neuropathies, Guillain-Barre syndrome (GBS), peripheral polyneuropathy, and nerve regeneration.

Provided are methods for treating or ameliorating one or more of the following features, symptoms, aspects, manifestations, or signs of multiple sclerosis, or other inflammatory disorder or condition of the nervous system: brain lesions, myelin lesions, demyelination, demyelinated plaques, visual disturbance, loss of balance or coordination, spasticity, sensory disturbances, incontinence, pain, weakness, fatigue, paralysis, cognitive impairment, bradyphrenia, diplopia, optic neuritis, paresthesia, gait ataxia, fatigue. Uhtoff's symptom, neuralgia, aphasia, apraxia, seizures, visual-field loss, dementia, extrapyramidal phenomena, depression, sense of well-being, or other emotional symptoms, chronic progressive myelopathy, and a symptom detected by magnetic resonance imaging (MRI), including gadolinium-enhancing lesions, evoked potential recordings, or examination of cerebrospinal fluid. See. e.g., Kenealy et al. (2003) J. Neuroimmunol. 143:7-12; Noseworthv et al. (2000) New Engl. J. Med. 343:938-952; Miller et al. (2003) New Engl. J. Med. 348:15-23; Chang et al. (2002) New Engl. J. Med. 346:165-173; Bruck and Stadelmann (2003) Neural. Sci. 24 Suppl.5:S265-S267.

Moreover, the present invention provides methods for treating and diagnosing inflammatory bowel disorders, e.g., Crohn's disease, ulcerative colitis, celiac disease, and irritable bowel syndrome. Provided are methods for treating or ameliorating one or more of the following symptoms, aspects, manifestations, or signs of an inflammatory bowel disorder: malabsorption of food, altered bowel motility, infection, fever, abdominal pain, diarrhea, rectal bleeding, weight loss, signs of malnutrition, perianal disease, abdominal mass, and growth failure, as well as intestinal complications such as stricture, fistulas, toxic megacolon, perforation, and cancer, and including endoscopic findings, such as, friability, aphthous and linear ulcers, cobblestone appearance, pseudopolyps, and rectal involvement and, in addition, anti-yeast antibodies. See, e.g., Podolsky, supra; Hanauer, supra; Horwitz and Fisher, supra.

Also contemplated is treatment of inflammatory disorders such as psoriasis, atopic dermatitis, arthritis, including rheumatoid arthritis, osteoarthritis, and psoriatic arthritis, autoimmune disorders, such as systemic lupus erythematosus and type I diabetes, and proliferative disorders such as cancer. See, e.g., PCT patent application publications WO 04/081190; WO 04/071517: WO 00/53631; and WO 01/18051.

The IL-23R binding compounds of the present invention can also be used in combination with one or more antagonists of other cytokines (e.g. antibodies), including but not limited to, IL-23p19, IL-17A, IL-17F, TNF-α, IL-1β, IL-6 and TGF-β. See, e.g., Veldhoen (2006) Immunity 24:179-189; Dong (2006) Nat. Rev. Immunol. 6(4):329-333. In various embodiments, an IL-23R binding compound of the invention is administered before, concurrently with, or after administration of the another antagonist or antagonists, such as an anti-IL-17A antibody. In one embodiment, an IL-1.7A binding compound is used in treatment of the acute early phase of an adverse immune response (e.g. MS, Crohn's Disease) alone or in combination with an IL-23R. antagonist antibody of the present invention. In the latter case, the IL-17A binding compound may be gradually decreased and treatment with the antagonist of IL-23R alone is continued to maintain suppression of the adverse response. Alternatively, antagonists to IL-23p19, IL-1β, IL-6 and/or TGF-β may be administered concurrently, before or after an IL-23R binding compound of the present invention. See Cua and Kastelein (2006) Nat. Immunol. 7:557-559; Tato and O'Shea (2006) Nature 441:166-168; Iwakura and Ishigame (2006) J. Clin. Invest. 116:1218-1222:

The broad scope of this invention is best understood with reference to the following examples, which are not intended to limit the inventions to the specific embodiments. The specific embodiments described herein are offered by way of example only, and the invention is to be limited by the terms of the appended claims, along with the full scope of equivalents to which such claims are entitled.

### EXAMPLES

### Example 1

### General Methods

Standard methods in molecular biology are described. Maniatis et al. (1982) Molecular Cloning, A Laboratory Manual, Cold Spring Harbor Laboratory Press, Cold Spring Harbor, N.Y.; Sambrook and Russell (2001) Molecular Cloning, 3^{rd} ed., Cold Spring Harbor Laboratory Press, Cold Spring Harbor, N.Y.; Wu (1993) Recombinant DNA, Vol. 217, Academic Press, San Diego, Calif. Standard methods also appear in Ausbel et al. (2001) Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described. Coligan et al. (2000) Current Protocols in Protein Science, Vol. 1, John Wiley and Sons, Inc., New York. Chemical analysis, chemical modification, post-translational modification, production of fusion proteins, glycosylation of proteins are described. See, e.g., Coligan et al. (2000) Current Protocols in Protein Science, Vol. 2, John Wiley and Sons, Inc., New York; Ausubel et al. (2001) Current Protocols in Molecular Biology, Vol. 3, John Wiley and Sons, Inc., NY, N.Y., pp. 16.0.5-16.22.17; Sigma-Aldrich, Co. (2001) Products for Life Science Research, St. Louis, Mo.; pp. 45-89; Amersham. Pharmacia Biotech (2001) BioDirectory, Piscataway, N.J., pp. 384-391. Production, purification, and fragmentation of polyclonal and monoclonal antibodies are described. Coligan et al. (2001) Current Protocols in Immunology, Vol. 1, John Wiley and Sons, Inc., New York; Harlow and Lane (1999) Using Antibodies, Cold Spring Harbor Laboratory Press, Cold Spring Harbor, N.Y.; Harlow and Lane, supra, Standard techniques for characterizing ligand/receptor interactions are available. See, e.g., Coligan et al. (2001) Current Protocols in Immunology, Vol. 4, John Wiley, Inc., New York.

Methods for flow cytometry, including fluorescence activated cell sorting detection systems (FACS®), are available. See, e.g., Owens et al. (1994) Flow Cytometry Principles for Clinical Laboratory Practice, John Wiley and Sons, Hoboken, N.J.; Givan (2001) Flow Cytometry, 2^{nd} ed.; Wiley-Liss, Hoboken, N.J.; Shapiro (2003) Practical Flow Cytometry, John Wiley and Sons, Hoboken, N.J. Fluorescent reagents suitable for modifying nucleic acids, including nucleic acid primers and probes, polypeptides, and antibodies, for use, e.g., as diagnostic. reagents, are available. Molecular Probes (2003) Catalogue, Molecular Probes, Inc., Eugene, Oreg.; Sigma-Aldrich (2003) Catalogue, St. Louis, Mo.

Standard methods of histology of the immune system are described. See, e.g,, Muller-Harmelink (ed.) (1986) Human Thymus: Histopathology and Pathology, Springer Verlag, New York, N.Y.; Hiatt, et al. (2000) Color Atlas of Histology, Lippincott, Williams, and Wilkins, Phila., Pa.; Louis, et al. (2002) Basic Histology Text and Atlas, McGraw-Hill, New York, N.Y.

Software packages and databases for determining, e.g., antigenic. fragments, leader sequences, protein folding, functional domains, glycosylation sites, and sequence alignments, are available. See, e.g., GenBank, Vector NTI® Suite (Informax, Inc, Bethesda, Md.); GCG Wisconsin Package (Accelrys, Inc., San Diego, Calif.); DeCypher® (TimeLogic Corp., Crystal Bay, Nev.); Menne et al. (2000) Bioinformatics 16: 741-742; Menne et al. (2000) Bioinformatics Applications Note 16:741-742; Wren et al. (2002) Comput. Methods Programs Biomed. 68:177-181; von Heijne (1983) Eur. J. Biochem. 133:17-21; von Heijne (1986) Nucleic Acids Res. 14:4683-4690.

### Example 2

### Generation and initial Humanization of Anti-human IL-23R Antibody 8B10

Anti-human IL-23R antibodies are generated by immunizing rats with the extracellular domain of human-IL-23R (residues 24-353 of SEQ ID NO: 9) as an Ig (human IgG1 Fc) fusion protein. Monoclonal antibodies are then prepared by standard methods.

The humanization of antibodies is described generally, e.g., in PCT patent application publications WO 2005/047324 and WO 2005/047326. Exemplary humanized heavy- and light-chain variable domain sequences are provided at FIGS. 1 and 2, respectively, and in the Sequence Listing.

Briefly, the amino acid sequence of the non-human VH domain (SEQ ID NO: 1) is compared to a group of three human VH germline amino acid sequences; one representative from each of subgroups IGHV1. IGHV3 and IGHV4. The VH subgroups are listed in M. -P. Lefranc (2001) “Nomenclature of the Human Immunoglobulin Heavy (IGH) Genes”, Experimental and Clinical Immunogenetics 18:100-116. The framework sequences of the human germline sequence with the closest match are used to construct a humanized VH domain.

Rat anti-huIL-23R antibody 8B10 is of the kappa subclass of VL. The amino acid sequence of the non-human VL domain (SEQ ID NO: 2) is compared to a group of four human VL kappa germline amino acid sequences. The group of four is comprised of one representative from each of four established human VL subgroups listed in V. Barbie & M. -P. Lefranc (1998) “The Human Immunoglobulin Kappa Variable (IGKV) Genes and Joining (IGKJ) Segments”, Experimental and Clinical Immunogenetics 15:171-183 and M. -P. Lefranc (2001) “Nomenclature of the Human Immunoglobulin Kappa (IGK) Genes”, Experimental and Clinical Immunogenetics 18:161-174. The four subgroups also correspond to the four subgroups listed in Kabat et al. (1991—5th Ed.) “Sequences of Proteins of Immunological Interest”, U.S. Department of Health and Human Services, NIH Pub. 91-3242, pp. 103-130. The framework sequence of the human germline sequence with the closest match is used to construct a humanized VL domain.

Once a candidate humanized antibody variable domain sequence is generated it can be further optimized. Serine residues may be replaced to reduce the likelihood of deamidation of the preceding asparagine residue. See Bischoff & Kolbe (1994) J. Chromatog. 662:261. The humanized heavy chain is then scrutinized to find positions at which the humanized framework sequences differed from the parental (rodent) framework sequences, which positions are considered likely causes of the reduced affinity. See, e.g., Tramontano et al. (1990) J. Mol. Biol. 215:175. (Note that unless otherwise indicated, sequence numbering in this patent application relates to the numbering of the sequence listing, and not to alternative numbering schemes such as the Kabat system.)

Once the target amino acid sequences of the variable heavy and light chains are determined, plasmids encoding the full-length humanized antibody may be generated.

Plasmid sequences may be altered using Kunkel mutagenesis (see, e.g., Kunkel T A. (1985) Proc. Natl. Acad. Sci. U.S.A. 82:488-492) to change the DNA sequence to the target humanized antibody sequences. Simultaneously, codon optimization may be performed to provide for potentially optimal expression. Sequence optimization may also be performed by commercial vendors, such as GENEART, Inc., Toronto, ON, Canada.

A humanized form of rat antibody clone 8B10 is constructed using human heavy chain sequence from subgroup III (germline sequence DP-46). The sequence of the resulting humanized heavy chain variable domain (hu8B10) is provided at SEQ ID NO: 11. Variants of hu8B10 heavy chain variable domain may be created to improve binding affinity or otherwise improve the properties of the resulting antibody. For example, the asparagine residue at position 30 of SEQ ID NO: 11 (in CDRH1) may be changed to threonine (N30T) to attempt to improve binding affinity. Framework residues may also be altered, such as residues A24, E46, W47, N76, N79, L81. or R100. Exemplary framework changes include E46D and W47L.

Light chain variable domain sequences are also constructed from rat antibody clone 8B10. A hu8B10 light chain variable domain sequence comprising the CDR sequences of the parental rat antibody and human light chain framework sequences from light chain subgroup kappa-1 (germline sequence Z-012) is provided at SEQ ID NO: 12.

### Example 3

### Determining the Equilibrium Dissociation Constant (K_{d}) for Anti-human IL-23R Antibodies Using KinExA Technology

The equilibrium dissociation constants (K_{d}) for anti human IL-23R antibodies are determined using the KinExA. 3000 instrument. Sapidyne Instruments Inc., Boise Id., USA. KinExA uses the principle of the Kinetic Exclusion Assay method based on measuring the concentration of uncomplexed antibody in a mixture of antibody, antigen and antibody-antigen complex. The concentration of free antibody is measured by exposing the mixture to a solid-phase immobilized antigen for a very brief period of time. In practice, this is accomplished by flowing the solution phase antigen-antibody mixture past antigen-coated particles trapped in a flow cell. Data generated by the instrument are analyzed using custom software. Equilibrium constants are calculated using a mathematical theory based on the lowing assumptions:
1. The binding follows the reversible binding equation for equilibrium:

kₒₙ[Ab] [Ag]=k_{off}[AbAg]
2. Antibody and antigen bind 1:1 and total antibody equals antigen-antibody complex plus free antibody.
3. Instrument signal is linearly related to free antibody concentration.

PMMA particles (Sapidyne, Cat No. 440198) are coated with biotinylated IL-23R (or a fragment thereof, such as the extracellular domain) according to Sapidyne “Protocol for coating PMMA particles with biotinylated ligands having short or nonexistent linker arms.” EZ-link TFP PEO-biotin (Pierce, Cat. No. 21219) is used to make biotinylated IL-23R, as per the manufacturer's recommendations (Pierce bulletin 0874).

Antibody r8B10 showed a K_{d} of 42 pM for binding to human IL-23R as determined by KinExA analysis.

### Example 4

### Determining the Equilibrium Dissociation Constant (K_{d}) for Humanized Anti-human IL-23R Antibodies Using BIAcore® Label-Free Interaction Analysis System Technology

BIAcore® label-free interaction analysis system determinations are performed essentially as described at Example 4 of U.S. Patent Application Publication No. 2007/0048315, which is hereby incorporated by reference. Briefly, ligands (anti-IL-23R mAbs) are immobilized on a BIAcore® label-free interaction analysis system CM5 sensor chip using standard amine-coupling procedure. Kinetic constants for the various interactions are determined using BIAevaluation software 3.1. The K_{d} is determined using the calculated dissociation and association rate constants.

Antibody r8B10 exhibited a K_{d} of approximately 300 pM for human IL-23R when assessed using the assay.

### Example 5

### Proliferation Bioassays for the Assessment of Neutralizing Anti-IL-23R Antibodies

The ability of a monoclonal antibody to biologically neutralize IL-23/IL-23R is assessed by the application of short-term proliferation bioassays that employ cells that express recombinant IL-23 receptors. The transfectara Ba/F3-2.21o cells proliferate in response to human IL-23 and the response can be inhibited by a neutralizing anti-IL-23R antibody. The concentration of IL-23 chosen for the assay is selected to be within the linear region of the dose-response curve, near plateau and above EC50. Proliferation, or lack thereof, is measured by calorimetric means using Alamar Blue, a growth indicator dye based on detection of metabolic activity. The ability of an antibody to neutralize IL-23/IL-23R is assessed by its IC50 value, or concentration of antibody that induces half-maximal inhibition of IL-23 proliferation.

The assay is performed essentially as follows. Ba/F3 transfectants are maintained in RPMI-1640 medium, 10% fetal calf serum, 50 μM 2-mercaptoethanol, 2 mM L-Glutamine. 50 μg/mL penicillin-streptomycin, and 10 ng/mL mouse IL-3. Proliferation bioassays are performed in RPMI-1640 medium, 10% fetal calf serum, 50 μM 2-mercaptoethanol, 2 mM L-Glutamine, and 50 μg/mL penicillin-streptomycin.

Assays are performed in 96-well flat bottom plates (Falcon 3072 or similar) in 150 μL per well. Both IL-23 and anti-IL-23R antibodies are added at a series of concentrations, e.g. 1:3 serial dilutions. Titrations of the anti-IL-23R antibody of interest are optionally pre-incubated with cells prior to addition of IL-23. Bioassay plates are incubated in a humidified tissue culture chamber (37C, 5% CO₂) for 40-48 hr. At the end of the culture time, Alamar Blue (Biosource Cat #DAL1100) is added at 16.5 μL/well and allowed to develop for 5-12 hours. Absorbance is then read at 570 nm and 600 nm (VERSAmax Microplate Reader, Molecular Probes, Eugene, Oreg., USA), and an OD₅₇₀₋₆₀₀ is obtained. Duplicates are run for each sample. Absorbance is plotted against cytokine or antibody concentration using GraphPad Prism® 3.0 software (Graphpad Software Inc., San Diego, Calif., USA), and IC50 values are determined using non-linear regression (curve fit) of sigmoidal dose-response.

### Example 6

### Mouse Splenocyte Assay for IL-23 Based on IL-17 Production

The biological activity of anti-IL-23R antibodies of the present invention may be assessed using the mouse splenocyte assay essentially as described in Aggarwal et al. (2003) J. Biol. Chem. 278:1910 and Stumhofer et al. (2006) Nature Immunol. 7:937. The mouse splenocyte assay measures the activity of IL-23 in a sample as a level of IL-17 production by murine splenocytes. The inhibitory activity of anti-IL-23R antibodies is then assessed by determining the concentration of antibody necessary to reduce the IL-23/IL-23R activity in a given sample by 50% (the IC50). The IC50 as measured by this assay is greater than or equal to the equilibrium dissociation binding constant (K_{d}), i.e. the K_{d} may be equal to or lower than the IC50. As always, lower IC50 and K_{d} values reflect higher activities and affinities.

Briefly, spleens are obtained from 8-12 wk old female C57BL/6J mice (Jackson Laboratories, Bar Harbor, Me., USA). Spleens are ground, pelleted twice, and filtered through a cell strainer (70 μm nylon). The recovered cells are cultured in 96-well plates (4×10⁵ cells/well) in the presence of human IL-23 (10 ng/ml, −170 pM) and mouse-anti-CD3e antibodies (1 μg/ml) (BD Pharmingen, Franklin Lakes, N.J., USA), with or without the anti-IL-23R antibody to be assayed. Anti IL-23R antibodies are added at a series of 3-fold dilutions. Cells are cultured for 72 hours, pelleted, and the supernatant is assayed for IL-17 levels by sandwich ELISA.

IL-17 ELISA is performed as follows. Plates are coated with a capture anti-IL-17 antibody (100 ng/well) overnight at 4° C., washed and blocked. Samples and standards are added and incubated for two hours at room temperature with shaking. Plates are washed, and a biotinylated anti-IL-17 detection antibody (100 ng/well) is added and incubated for one hour at room temperature with shaking. The capture and detection antibodies are different antibodies that both bind to mouse IL-17 but do not cross-block. Plates are washed, and bound detection antibody is detected using streptavidin-HRP (horseradish peroxidase) and TMB (3,3′,5,5′-tetramethylbenzidine). The plate is then read at 450-650 nm and the concentration of IL-17 in samples is calculated by comparison with standards.

### Example 7

### Optimization of Anti-IL-23R Antibody hu8B10

An improved variant of humanized anti-IL-23R antibody 8B10 (Example 2, supra) was generated, starting with the heavy and light chain sequences of SEQ ID NOs: 11 and 12. The resulting antibody is referred to as hu8B10-b. Sequences are provided at FIGS. 1 and 2, and SEQ ID NOs: 11-17. The CDR sequences of antibody 8B10-b and antibody 8B10 are identical other than CDRL1, although the framework regions differ on both the heavy and light chains. The antibody may be prepared from mammalian cells using a vector harboring DNA sequences encoding the heavy and light chains, for which amino acid sequences are provided at SEQ ID NOs: 16 and 17, respectively.

As illustrated in FIG. 1, hu8B10-b VH differs from hu8B10 VH at three positions (E46D, W47L and R100V), In each case the change reverts a human framework (DP-46) residue to the residue in the parental rat framework.

As illustrated in FIG. 2, hu8B10-b VL differs from hu8B10 VL at three positions within CDRL1 (L24R, E27Q and N31T) and at one framework residue (F71Y). The F71Y change reverts the human framework (Z-012) residue to the residue in the parental rat framework region. The L24R and E27Q changes replace the rat CDR residue with the corresponding human sequence from germline Z-012.

The N31T change was introduced as a result of affinity maturation experiments, as follows. An E. coli expression vector for expression into the periplasmic space was constructed encoding a chimeric Fab fragment of rat antibody 8B10 comprising rat variable domains, a human constant light chain domain (C_{L}) and a human constant heavy chain domain C_{H}1. Mutations were introduced using a QuikChange® site directed mutagenesis kit (Stratagene Inc., LaJolla, Calif., USA). Primers were designed to replace selected positions with either alanine, or by all 20 amino acids, using the NNK codon approach (N includes an equimolar mix of A/G/C/T and K includes only G/T). To reduce background, alanine mutants at each position were used as the template for the randomization step. Unpurified periplasmic extracts were then tested for human IL-23R binding activity using an electrochemiluminescence assay with biotinylated huIL-23R-IgG and goat anti-huFab-Ori-Tag for detection. The area under the curve was used to compare the binding activity of sequence variants to the original parental 8B10 Fab. Data were fitted using GraphPad Prism® 3.0 software (Graphpad Software inc., San Diego, Calif. USA). Results are shown at Table 2.

TABLE 2
Mutagenesis of anti-IL23R Antibody 8B10

| | | Binding (% of w.t.) |
|---|---|---|
| Region | Mutation | (mean ± SD) |
| VH | W47L | 0 |
| | A49G | 79.0 ± 5.8 |
| | R74A | 52.6 ± 1.7 |
| | H101A | 29.1 ± 6.3 |
| CDR H3 | H102A | 84.2 ± 6.6 |
| | S103A | 65.7 ± 4.0 |
| | D104A | 1.5 ± 0.5 |
| | Y105A | 18.3 ± 0.9 |
| | F106A | 71.1 ± 5.1 |
| | E107A | 61.0 ± 2.6 |
| | H102W | 129.2 ± 16.0 |
| | H102W | 101.7 ± 8.1 |
| | D50A | 67.3 ± 2.9 |
| | I51A | 123.3 ± 2.9 |
| | N52A | 38.7 ± 4.7 |
| CDR H2 | S53A | 88.7 ± 2.8 |
| | K54A | 71.1 ± 8.2 |
| | S55A | 104.6 ± 7.4 |
| | Y56A | 0.2 ± 0.2 |
| | Y58G | 53.0 ± 4.6 |
| | Y61A | 41.7 ± 3.3 |
| | S31A | 100.5 ± 14.5 |
| CDR H1 | Y32A | 76.3 ± 4.8 |
| | G33A | 27.4 ± 1.4 |
| VL | F71Y | 33.5 ± 0.7 |
| | D91A | 5.6 ± 0.1 |
| CDR L3 | S92A | 68.4 ± 5.7 |
| | E93A | 62.8 ± 10.2 |
| | Y94A | 0 |
| | Y49A | 62.4 ± 0.1 |
| CDR L2 | H50A | 62.16 ± 7.1 |
| | S53A | 85.0 ± 6.8 |
| CDR L1 | E27A | 96.2 ± 4.9 |
| | D28A | 93.1 ± 0.1 |
| | Y30A | 3.2 ± 0.4 |
| | N31A | 116.9 ± 8.8 |
| | N31del | 0 |
| | N32A | 13.4 ± 11.4 |
| | N31T | 128.7 ± 4.5 |

Table 2 provides structure activity relationship data for antibody 8B10. For example, several variants are shown to have greatly decreased binding to human IL-23R, such as heavy chain variable domain variants W47L, D104A and Y56A, and light chain variable domain variants D91A, Y94A, Y30A and N31del. Other variants exhibit somewhat improved affinity compared to the original starting sequence, such as heavy chain variable domain variants H102W, I51A, S55A and S31A, and light chain variable domain variants N31A and N31T.

The N31T variant was reformatted to a full-length chimeric antibody, expressed in (human) HEK293 cells, and purified. K_{d} was determined by the KinExA method (see Example 3, supra), wherein the K_{d} for N31T was 8, as compared with 25 pM for the original sequence. Accordingly, the N31T variant exhibits a three-fold improvement in binding affinity.

### Example 8

### Characterization of Anti-IL-23R Antibody hu8B 10-b and Variants

Experiments were performed to determine the binding affinity and bioactivity of the improved hu8B10-b antibody (and variants thereof) as follows,

Several sequence variants intended to increase the chemical stability of hu8B10-b were produced to evaluate their effects on binding affinity. K_{d} values are provided at Table 3. Binding to human IL-23R was determined using the BIAcore® label-free interaction analysis system (surface plasmon resonance), essentially as described in Example 4 (supra). Chimeric forms, designated as “chim,” refers to rat variable domains linked to human IgG1 heavy chain and human kappa light chain constant domains, e.g., as presented at SEQ ID NOs: 16 and 17, respectively, or as at SEQ ID NOs: 22 and 23, respectively. Sequences for heavy and light chain variable domains are provided at FIGS. 1 and 2, and in the Sequence Listing (as described at Table 4). Antibodies incorporate the sequence changes recited in the table (“Variant(s)”) for the respective heavy and light chains. Data arc presented as mean±standard deviation for multiple determinations, and without error estimates for single determinations.

TABLE 3
Comparison of anti-IL-23R Antibody 8B10-b Variants

| Heavy/Light Chain | | K_{d} (pM) |
|---|---|---|
| Variable Domains | Variant(s) | (mean ± SD) |
| VH: r8B10 | — | 270 ± 43 |
| VL: r8B10 | — | |
| VH: chim8B10 | — | 345 ± 56 |
| VL: chim8B10 | — | |
| VH: hu8B10 | — | no binding |
| VL: hu8B10 | N31T | |
| VH: hu8B10-b | — | 5,383 ± 587 |
| VL: hu8B10 | N31T | |
| VH: 8B10-b | — | 154 ± 24 |
| VL: 8B10-b | — | |
| VH: 8B10-b | — | 19,177 |
| VL: 8B10-b | N32S | |
| VH: 8B10-b | — | 187 |
| VL: 8B10-b | D56S | |
| VH: 8B10-b | — | 179 |
| VL: 8B10-b | D56E | |
| VH: 8B10-b | N30S | 168 |
| VL: 8B10-b | — | |
| VH: 8B10-b | N30D | 114 |
| VL: 8B10-b | — | |
| VH: 8B10-b | N52S | 444 |
| VL: 8B10-b | — | |
| VH: 8B10-b | N52D | no binding |
| VL: 8B10-b | — | |
| VH: 8B10-b | N57S | 382 |
| VL: 8B10-b | — | |
| VH: 8B10-b | N57D | 220 |
| VL: 8B10-b | — | |
| VH: 8B10-c | — | 180 ± 10 |
| VL: 8B10-c | — | |

Table 3 provides structure activity relationship data for antibody 8B10-b. Several variants were found to essentially retain binding affinity, such as VL D56S and D56E, and VH N30S, N30D, N52S, N57S, N57D.

The biological activity of hu8B10-b may also be assessed using a human splenocyte assay, essentially as described in Example 6 (supra) with the exception that splenocytes are obtained from human spleens rather than mouse, no anti-CD3e antibody is used, and that IFN-γ is the readout rather than IL-17. The assay measures the activity of IL-23 in a sample by determining the level of IFN-γ production by human primary splenocytes. Human splenocytes are exposed to human IL-23 (170 pM.) in the presence of various concentrations of anti-IL-23R antibody hu8B10-b, or in the absence of the antibody. IFN-γ is detected by sandwich ELISA.

The biological activity of hu8B10-b may be further assessed using a KIT225 STAT-3 phosphorylation assay, essentially as described in Parham et al. (2002) J. Immunol. 168:5699, Human KIT225 cells, a leukemic T cell line, are stimulated with 138 pM human IL-23 in the presence of various concentrations of anti-IL-23R antibody hu8B10-b, or in the absence of the antibody. IL-23 activity is measured by detecting the level of STAT3 phosphorylation.

One variant of 8B10-b, having light chain D56S and heavy chain N30S amino acid substitutions, was named 8B10-c. Heavy and light chain variable domains are provided at SEQ ID NOs: 22 and 23, respectively. Full length heavy and light chains (with human IgG1 heavy chain and human kappa light chain constant domains) are provided at SEQ ID NOs: 24 and 25, respectively. A human IgG4 version of the 8B10-c heavy chain is provided at SEQ ID NO: 26.

Table 4 provides a brief description of the sequences in the sequence listing.

TABLE 4
Sequence Identifiers

| SEQ ID NO: | Description |
|---|---|
| 1 | 8B10 Heavy Chain Variable |
| 2 | 8B10 Light Chain Variable |
| 3 | 8B10 CDRH1 |
| 4 | 8B10 CDRH2 |
| 5 | 8B10 CDRH3 |
| 6 | 8B10 CDRL1 |
| 7 | 8B10 CDRL2 |
| 8 | 8B10 CDRL3 |
| 9 | Human IL-23R |
| 10 | Human IL-12Rβ1 |
| 11 | hu8B10 Heavy Chain Variable |
| 12 | hu8B10 Light Chain Variable |
| 13 | hu8B10-b Heavy Chain Variable |
| 14 | hu8B10-b Light Chain Variable |
| 15 | hu8B10-b CDRL1 |
| 16 | hu8B10-b Heavy Chain (IgG1) |
| 17 | hu8B10-b Light Chain |
| 18 | hu8B10 Heavy Chain Signal Sequence |
| 19 | hu8B10 Light Chain Signal Sequence |
| 20 | hu8B10-c CDRH1 |
| 21 | hu8B10-c CDRL2 |
| 22 | hu8B10-c Heavy Chain Variable |
| 23 | hu8B10-c Light Chain Variable |
| 24 | hu8B10-c Heavy Chain (IgG1) |
| 25 | hu8B10-c Light Chain |
| 26 | hu8B10-b Heavy Chain (IgG4) |
| 27 | hu8B10-b Heavy Chain (IgG4) DNA |
| 28 | hu8B10-c Light Chain DNA |

What is claimed is:

1. A binding compound that binds to human IL-23R comprising an antibody light chain variable domain, or antigen binding fragment thereof, comprising:
a) the CDRL1 sequence of SEQ ID NO: 15; or
b) the CDRL2 sequence of SEQ ID NO: 21.

2. The binding compound of claim 1(a) further comprising a CDR sequence selected from the group consisting of SEQ ID NOs: 7 and 8.

3. The binding compound of claim 2 comprising the CDR sequences SEQ ID NOs: 7 and 8.

4. The binding compound of claim 1 further comprising an antibody heavy chain variable domain, or antigen binding fragment thereof, comprising one or more CDR sequences selected from the group consisting of SEQ ID NOs: 3, 4, 5 and 20.

5. The binding compound of claim 4 comprising an antibody heavy chain variable domain, or antigen binding fragment thereof, comprising two or more CDR sequences selected from the group consisting of SEQ ID NOs: 3, 4, 5 and 20.

6. The binding compound of claim 5 comprising an antibody heavy chain variable domain, or antigen binding fragment thereof, comprising CDR sequences SEQ ID NOs: 3-5.

7. The binding compound of claim 2 comprising an antibody heavy chain variable domain, or antigen binding fragment thereof, comprising CDR sequences SEQ ID NOs: 3-5,

8. A binding compound that binds to human IL-23R comprising:
i) an antibody light chain variable domain, or antigen binding fragment thereof, comprising CDR sequences SEQ ID NOs: 15, 7 and 8: and
ii) an antibody heavy chain variable domain, or antigen binding fragment thereof, comprising CDR sequences SEQ ID NOs: 3-5.

9. The binding compound of claim 8 wherein the tight chain variable domain, or antigen binding fragment thereof, comprises SEQ ID NO: 14.

10. The binding compound of claim 8 wherein the heavy chain variable domain, or antigen binding fragment thereof, comprises SEQ ID NO: 13.

11. The binding compound of claim 9 wherein the heavy chain variable domain, or antigen binding fragment thereof, comprises SEQ ID NO: 13.

12. A binding compound that binds to human IL-23R, comprising:
i) an antibody light chain variable domain, or antigen binding fragment thereof, comprising SEQ ID NO: 14, or a variant thereof; and
ii) an antibody heavy chain variable domain, or antigen binding fragment thereof, comprising SEQ ID NO: 13 or a variant thereof;
wherein each variant comprises up to 20 conservatively modified amino acid substitutions.

13. A binding compound that binds to human IL-23R, comprising:
i) an antibody light chain variable domain, or antigen binding fragment thereof, consisting essentially of SEQ ID NO: 14; and
ii) an antibody heavy chain variable domain, or antigen binding fragment thereof, consisting essentially of SEQ ID NO: 13.

14. A binding compound that binds to human IL-23R, comprising:
i) a light chain variable domain having at least 90% homology to SEQ ID NO: 14; and
a heavy chain variable domain having at least 90% homology to SEQ ID NO: 13.

15. An antibody, or antigen binding fragment thereof, that is able to compete with a binding compound to human IL-23R in a cross-blocking assay, wherein the binding compound comprises:
i) a light chain variable domain comprising SEQ ID NO: 14; and
ii) a heavy chain variable domain comprising SEQ ID NO: 13.

16. An isolated nucleic acid encoding at least one of the light chain variable domain or the heavy chain variable domain of the binding compound of claim 11.

17. An expression vector comprising the nucleic acid of claim 16 operably linked to control sequences that are recognized by a host cell.

18. A host cell comprising the expression vector of claim 17.

19. A method of producing a polypeptide comprising:
i) culturing the host cell of claim 18 in culture medium under conditions wherein the nucleic acid sequence is expressed, thereby producing at least one of the polypeptides comprising the light and heavy chain variable domains; and
ii) recovering the polypeptide from the host cell or culture medium.

20. The binding compound of claim 11, further comprising a heavy chain constant region comprising a γ1 human heavy chain constant region or a variant thereof, wherein the constant region variant comprises up to 20 conservatively modified amino acid substitutions.

21. The binding compound of claim 11, further comprising a heavy chain constant region comprising a γ4 human heavy chain constant region or a variant thereof, wherein the constant region variant comprises up to 20 conservatively modified amino acid substitutions.

22. The binding compound of claim 13, wherein the binding compound is an antibody fragment selected from the group consisting of Fab, Fab′, Fab′-SH, Fv, scFv, F(ab′)₂, and a diabody.

23. A method of suppressing an immune response in a human subject comprising administering to a subject in need thereof an antibody of claim 11, or a antigen binding fragment thereof, in an amount effective to block the biological activity of IL-23R.

24. The method of claim 23, wherein the biological activity is an inflammatory response.

25. The method of claim 23, wherein the biological activity is an autoimmune response.

26. The method of claim 23, wherein the subject has a disorder selected from the group consisting of arthritis, psoriasis, inflammatory bowel disease, multiple sclerosis, systemic lupus erythematosus and diabetes.

27. The method of claim 23, wherein the subject has cancer and the biological activity is a Th17 response.

28. The method of claim 23, further comprising administering an immunosuppressive or anti-inflammatory agent.

29. A pharmaceutical composition comprising the binding compound of claim 11 in combination with a pharmaceutically acceptable carrier or diluent.

30. A binding compound that binds to human IL-23R, comprising:
an antibody light chain comprising SEQ ID NO: 17; and
an antibody heavy chain comprising SEQ ID NO: 16.

31. A binding compound that binds to human IL-23R, comprising:
an antibody light chain consisting of SEQ ID NO: 17; and
an antibody heavy chain consisting of SEQ ID NO: 16.

32. An antibody, or antigen binding fragment thereof, that binds to human IL-23R, comprising:
i) a light chain variable domain omprising SEQ ID NO: 14 with no substitutions, or with the substitution D56S or D56E; and
ii) a heavy chain variable domain comprising SEQ ID NO: 13 with no substitutions, or with one or more substitutions selected from the group consisting of N30S. N30D, N52S, N57S and N57D.

33. The antibody or antigen binding fragment of claim 32 comprising:
i) a light chain variable domain comprising SEQ ID NO: 23; and
ii) a heavy chain variable domain comprising SEQ ID NO: 22.

34. The antibody of claim 33 comprising:
i) a light chain comprising SEQ ID NO: 25; and
ii) a heavy chain comprising SEQ ID NO: 24.

35. The antibody of claim 33 comprising:
a light chain comprising SEQ ID NO: 25; and
ii) a heavy chain comprising SEQ ID NO: 26.

36. An expression vector comprising a nucleic acid sequence encoding at least one of the light chain and the heavy chain of claim 33, operably linked to control sequences that are recognized by a host cell.

37. A host cell comprising the expression vector of claim 36.

38. A method of producing a polypeptide comprising:
i) culturing the host cell of claim 37 in culture medium under conditions wherein the nucleic acid sequence is expressed, thereby producing at least one of the light chain and the heavy chain; and
ii) recovering at least one of the light chain polypeptide and the heavy chain polypeptide from the host cell or culture medium.

39. A nucleic acid comprising the sequence of residues 58-1395 SEQ ID NO: 27.

40. A nucleic acid comprising the sequence of residues 58-699 of SEQ ID NO: 28.

41. A host cell comprising one or more expression vectors comprising the nucleic acids of claims 39 and 40.
